(19) United States
(12) Patent Application Publication
(10) Pub. No.: US 2011/0106076 A1
(43) Pub. Date: May 5, 2011

(54) MYOABLATION SYSTEM

(76) Inventor: Gregorio Hernandez Zendejas, Guadalajara (MX)

(21) Appl. No.: 12/611,939

(22) Filed: Nov. 4, 2009

(51) Int. Cl. A61B 18/14

(52) U.S. Cl. 606/42

(57) ABSTRACT

Myoablation system (FIG. 1) for modifying the facial dynamics. A hand piece (100) is provided for increasing the internal energy in a part of a facial muscle via the percutaneous route. A switch (116) for alternating between two or more circuit configurations connected to hand piece (100). A power supply (128) for providing power connected to switch (116). A muscle detector (118) for detecting muscle tissue connected to switch (116).

### TECHNICAL FIELD

This application relates to the field of surgery and, more particularly, to minimally invasive surgery.

### CROSS-REFERENCE TO RELATED APPLICATIONS

Not applicable.

### FEDERALLY SPONSORED RESEARCH

Not applicable.

### SEQUENCE LISTING OR PROGRAM

Not applicable.

### BACKGROUND

The facial muscles are a group of striated muscles innervated by the facial nerve that, among other things, control facial dynamics. Modification of the facial dynamics is beneficial in certain medical conditions. A well-established procedure for modifying the facial dynamics involves the surgical excision of a portion of a facial muscle (selective myectomy). Selective myectomy is used to treat certain medical conditions such as facial paralysis, and migraine (Baker D C: Facial palsy. In McCarthy J G (Ed): Plastic Surgery Vol. 3 The Face. Philadelphia: W.B. Saunders Company, 1990, p 2306; Guyuron B, Kriegler J S, Davis J, Amini S B: Comprehensive surgical treatment of migraine headaches. Plast Reconstr Surg 115:1, 2005). Selective myectomy or myotomy is critical to achieve improved aesthetic results in aesthetic facial surgery (Abramo A C, Dorta A A: Selective Myotomy in Forehead Endoscopy. Plast Reconstr Surg 112:873, 2003). Nevertheless selective myectomy involves surgical and anesthetic risks.

In an attempt to avoid these surgical and anesthetic risks, minimally invasive methods for reduce the function of the facial muscles have been proposed. These minimally invasive methods involve the application of either energy or a substance through the skin. The application of either energy or a substance through the skin can be percutaneous or transcutaneous. Percutaneous pertains to any medical procedure where access to inner organs or other tissue is done via needle-puncture of the skin, rather than by using an “open” approach where inner organs or tissue are exposed. Transcutaneous pertains to any medical procedure where access to inner organs or other tissue is done via the unbroken skin.

Percutaneous, and transcutaneous minimally invasive methods for reduce the function of the facial muscles have been proposed. One of those methods involves percutaneous botulin toxin injections. Botulin toxin paralyses muscles, reducing the facial muscles function (Bulstrode N W, Grobbelaar A O: Long-term prospective follow-up of botulinum toxin treatment for facial rhytides. Aesth Plast Surg 26:356-359, 2002). However, botulin toxin injections effect lasts a few months.

Implants for continuous in vivo release of a neurotoxin over a period ranging from several days to a few years have been proposed (U.S. Pat. No. 6,383,509 B1; U.S. Pat. No. 6,506,399 B2; U.S. Pat. No. 6,312,708 B1; U.S. Pat. No. 6,585,993 B2; U.S. Pat. No. 6,306,423 B1). These implants can contain botulin toxin. Nevertheless if for some reason the botulin toxin contained in such implants is released faster than expected, poisoning caused by botulin toxin may issue.

Percutaneous denervation of facial muscles to modify the facial dynamics has been reported (Hernandez Zendejas G, Guerrerosantos J: Percutaneous selective radio-frequency neuroablation in plastic surgery. Aesth Plast Surg 18:41-48, 1994; Utley D S, Goode R L: Radiofrequency ablation of the nerve to the corrugator muscle for elimination of glabellar furrowing. Arch Facial Plast Surg 1:46, 1999; U.S. Pat. No. 6,139,545; US 2005/0283148 A1; US 2007/0060921 A1; US 2007/0167943 A1). This technique involves the destruction of nerves by electro fulguration (charring) of tissues using high-energy radio frequency energy.

Transcutaneous methods and devices for treatment of a muscle have been proposed (US 2007/0255342 A1). Such methods involve applying an electrical impulse to facial nerves rather than to facial muscles. In theory if the nerves are deactivated, the muscles should be deactivated as well.

However, frequently the muscles regain function. Long-term results can be unpredictable because denervated facial muscles undergo reinnervation (Baker D C: Facial palsy. In McCarthy J G (Ed): Plastic Surgery Vol. 3 The Face. Philadelphia: W.B. Saunders Company, 1990, p 2255). Moreover, denervation of facial muscles is burdensome since facial muscles innervation is inconsistent (Schwember G, Rodriguez A: Anatomical surgical dissection of the extraparotid portion of the facial nerve. Plast Reconstr Surg 81:183, 1988; Caminer D M, Newman M I, Boyd J B: Angular nerve: New insights on innervation of the corrugator supercilii and procerus muscles. Plast Reconstr Aesth Surg 59:366, 2006). Therefore the main target should be the facial muscles rather than the facial nerves.

Transcutaneous energy delivering for creating a lesion in facial muscles to modify the facial dynamics has been proposed (US 2007/0032784 A1; US 2008/0071255 A1). Nevertheless this technique is not muscle-specific and creates a lesion not only in muscle tissue, but also in contiguous vital structures such as nerves, arteries, veins, and lymphatics. Moreover, long-term results can be unpredictable because the lesion created involves not only muscle tissue, but also contiguous vital structures such as nerves, arteries, veins, and lymphatics.

### SUMMARY

Myoablation system for modifying the facial dynamics. A hand piece is provided for increasing the internal energy in a part of a facial muscle via the percutaneous route. A switch for alternating between two or more circuit configurations connected to the hand piece. A power supply for providing power connected to the switch. A muscle detector for detecting muscle tissue connected to the switch.

### BRIEF DESCRIPTION OF THE DRAWINGS

A complete understanding of the present invention may be obtained by reference to the accompanying drawings, when considered in conjunction with the subsequent, detailed description, in which:

FIG. 1 is an overall view of a myoablation system of the first embodiment;

FIG. 2 is a partial view of the myoablation system shown in FIG. 1;

FIG. 3 is a schematic view of the myoablation system shown in FIG. 1;

FIG. 4 is an enlarged partial view of the myoablation system shown in FIG. 3;

FIG. 5 is a perspective view of the thermal needle electrode tip shown in FIG. 2;

FIG. 6 is a perspective sectional view of the thermal needle electrode tip shown in FIG. 5;

FIG. 7 is an enlarged view of the distal end of the thermal needle electrode tip shown in FIG. 5;

FIG. 8 is an enlarged sectional view of the distal end of the thermal needle electrode tip shown in FIG. 6;

FIG. 9A is a perspective view of an additional embodiment of the thermal needle electrode tip;

FIG. 9B is a perspective view of an additional embodiment of the thermal needle electrode tip;

FIG. 9C is a perspective view of an additional embodiment of the thermal needle electrode tip;

FIG. 9D is a perspective view of an additional embodiment of the thermal needle electrode tip;

FIG. 9E is a perspective view of an additional embodiment of the thermal needle electrode tip;

FIG. 9F is a perspective view of an additional embodiment of the thermal needle electrode tip;

FIG. 9G is a perspective view of an additional embodiment of the thermal needle electrode tip;

FIG. 9H is a perspective view of an additional embodiment of the thermal needle electrode tip;

FIG. 10 is a schematic view of a circuitry of the muscle detector shown in FIG. 1;

FIG. 11 is an operational view of the myoablation system shown in FIG. 1;

FIG. 12A is an operational view of the myoablation system shown in FIG. 1;

FIG. 12B is an operational view of the myoablation system shown in FIG. 1;

FIG. 13A is an operational view of the myoablation system shown in FIG. 1;

FIG. 13B is an operational view of the myoablation system shown in FIG. 1;

FIG. 14 is a schematic view of the facial muscles anatomy;

FIG. 15 is a schematic view of a local anesthesia procedure;

FIG. 16 is a schematic view of a myoablation procedure;

FIG. 17A is a schematic view of a myoablation procedure;

FIG. 17B is a schematic view of a myoablation procedure;

FIG. 18 is an overall view of an alternate embodiment of the myoablation system;

FIG. 19 is a partial view of the alternate embodiment of the myoablation system shown in FIG. 18;

FIG. 20 is a schematic view of the myoablation system shown in FIG. 18;

FIG. 21 is an enlarged partial view of the myoablation system shown in FIG. 20;

FIG. 22 is a perspective view of the radio frequency energy needle electrode tip shown in FIG. 19;

FIG. 23 is a perspective sectional view of the radio frequency energy needle electrode tip shown in FIG. 22;

FIG. 24 is an enlarged view of the distal end of the radio frequency energy needle electrode tip shown in FIG. 22;

FIG. 25 is an enlarged sectional view of the distal end of the radio frequency energy needle electrode tip shown in FIG. 23;

FIG. 26 is a schematic view of a circuitry of the radio frequency energy generator shown in FIG. 21;

FIG. 27A is a perspective view of an additional embodiment of the radio frequency energy needle electrode tip;

FIG. 27B is a perspective view of an additional embodiment of the radio frequency energy needle electrode tip;

FIG. 27C is a perspective view of an additional embodiment of the radio frequency energy needle electrode tip;

FIG. 27D is a perspective view of an additional embodiment of the radio frequency energy needle electrode tip;

FIG. 27E is a perspective view of an additional embodiment of the radio frequency energy needle electrode tip;

FIG. 27F is a perspective view of an additional embodiment of the radio frequency energy needle electrode tip;

FIG. 27G is a perspective view of an additional embodiment of the radio frequency energy needle electrode tip;

FIG. 27H is a perspective view of an additional embodiment of the radio frequency energy needle electrode tip;

FIG. 28 is an operational view of the alternate embodiment of the myoablation system shown in FIG. 18;

FIG. 29 is a schematic view of a myoablation procedure;

FIG. 30A is a schematic view of a myoablation procedure;

FIG. 30B is a schematic view of a myoablation procedure;

FIG. 31 is a perspective view of an alternate embodiment of the myoablation system;

FIG. 32 is a perspective view of the alternate embodiment of the myoablation system shown in FIG. 31;

FIG. 33 is a schematic sectional view of the alternate embodiment of the myoablation system shown in FIG. 31;

FIG. 34 is an enlarged sectional view of the alternate embodiment of the myoablation system shown in FIG. 33;

FIG. 35 is an operational view of the alternate embodiment of the myoablation system shown in FIG. 31;

FIG. 36 is a schematic view of a myoablation procedure;

FIG. 37A is a schematic view of the myoablation procedure;

FIG. 37B is a schematic view of the myoablation procedure;

FIG. 38 is a perspective view of an alternate embodiment of the myoablation system;

FIG. 39 is a perspective view of the alternate embodiment of the myoablation system shown in FIG. 38;

FIG. 40 is a schematic sectional view of the alternate embodiment of the myoablation system shown in FIG. 38;

FIG. 41 is an enlarged partial view of the alternate embodiment of the myoablation system shown in FIG. 40;

FIG. 42 is an operational view of the alternate embodiment of the myoablation system shown in FIG. 38;

FIG. 43 is a schematic view of the myoablation procedure;

FIG. 44A is a schematic view of the myoablation procedure;

FIG. 44B is a schematic view of the myoablation procedure;

FIG. 45A is a partial view of an alternate embodiment of the myoablation system;

FIG. 45B is a partial view of an alternate embodiment of the myoablation system;

FIG. 45C is a perspective view of an alternate embodiment of the myoablation system; and

FIG. 45D is a perspective view of an alternate embodiment of the myoablation system.

For purposes of clarity and brevity, like elements and components will bear the same designations and numbering throughout the Figures.

### GLOSSARY

Glossary of technical and medical terms.

Internal energy: The internal energy of a system is the total energy of all of the molecules in the system. Internal energy comprises the vibrational energy of molecules and of atoms within molecules, energy due to interaction between the atoms and molecules, and energy associated with the binding of atoms to form molecules.

Heat: A flow of energy between two objects or systems due to temperature difference between them.

PC: Personal computer.

Facial muscles. Group of striated muscles innervated by the facial nerve that, among other things, control facial expression.

Corrugator supercilii muscles: Skeletal muscles of the forehead that produce frowning and brow depression.

Muscle electrical signal: Electrical signal produced by muscle activity.

Contractile proteins actin and myosin: Muscle fibers contain certain protein molecules called actin and myosin. Actin and myosin are responsible for the actual muscle contraction.

Defunctionalized: Nonfunctional biological tissue.

Myoablation: Devitalize a facial muscle by increasing its internal energy via the percutaneous route (myo=muscle: a prefix used in biology to denote muscle; ablation=erosion: removal of a part of biological tissue; to remove or decrease something by the process of ablation).

Myoablation procedure: Series of steps taken to accomplish a myoablation

### DETAILED DESCRIPTION

### First Embodiment

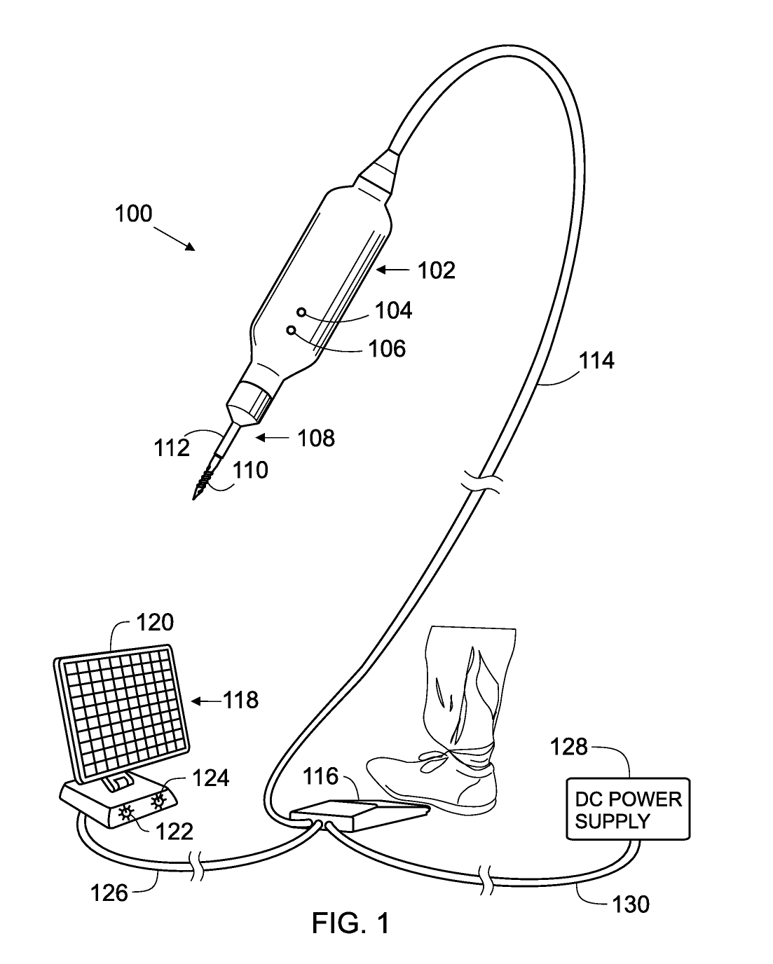

### FIGS. 1, 2, 3, 4, 5, 6, 7, 8, 9a, 9b, 9c, 9d, 9e, 9f, 9G, 9H, 10

A prototype of the myoablation system was built, and tested by the inventor.

FIG. 1 shows an overall view of the myoablation system of the first embodiment. The myoablation system of FIG. 1 has a hand piece 100 comprising a handle 102 and a detachable thermal needle electrode tip 108. Handle 102 has an energize indicator 104 and a time indicator 106. Thermal needle electrode tip 108 has a resistive heating element 110 and a percutaneous return electrode 112.

Hand piece 100 is operably connected by a mix cable 114 to a switch 116. Switch 116 is operably connected by an input cable 126 to a muscle detector 118. Muscle detector 118 has a graphic display 120, a gain control 122, and a sweep time control 124. In addition, switch 116 is operably connected by a power cable 130 to a power supply 128.

Hand piece 100 is provided for increasing the internal energy in a part of a facial muscle via the percutaneous route.

I contemplate that handle 102 of this embodiment is made of plastic, but others materials are also suitable.

Energize indicator 104 is provided for indicating actual delivering of energy to thermal needle electrode tip 108. I presently contemplate that energize indicator 104 of this embodiment comprises a green light emitting diode (LED) connected to a 100 ohms resistor, but others components are also suitable.

Time indicator 106 is provided for measuring time of actual delivering of energy to thermal needle electrode tip 108. I presently propose that time indicator 106 of this embodiment consists of a yellow blinking LED with a 2 Hz blinking rate available from NTE Electronic Inc. of Bloomfield, N.J. (www.nteinc.com). Notwithstanding others components for measuring or indicating time are also suitable, such as a clock, watch, stopwatch, etc.

Switch 116 is provided for alternating between two or more circuit configurations. I presently contemplate that switch 116 of this embodiment consists of a triple pole triple throw momentary (push-to-make) foot switch. However others components for alternating between two or more circuit configurations are also suitable, such as a microswitch, reed switch, tilt switch, transistor, relay, computer-controlled switching mechanism, optoelectronic mechanism, nanotechnology mechanism, molecular mechanism, etc.

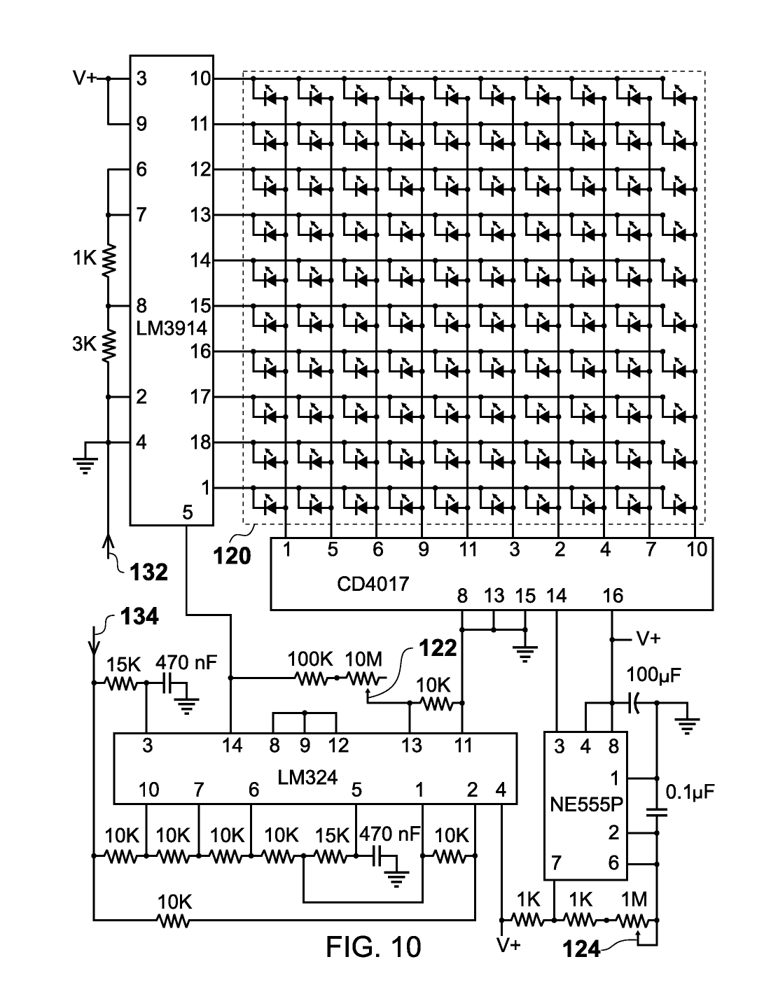

Muscle detector 118 is provided for detecting muscle tissue. I presently propose an electronic version of muscle detector 118 comprising a circuitry shown in FIG. 10. However it can comprise a combination of a computer-based oscilloscope and a muscle stimulator. Notwithstanding others components for detecting muscle tissue are also suitable, such as an electromyograph, an oscilloscope, a computer-based oscilloscope, a muscle stimulator, an endoscope, etc. I contemplate that muscle detector 118 of this embodiment has its own power source (not shown).

Power supply 128 is provided for supplying power to hand piece 100. I presently propose that power supply 128 of this embodiment consists of a 2 Amps regulated DC power source with a variable output range of 4.5 to 9 volt DC. Nevertheless other power sources are also suitable, such as a battery, electrochemical cell, thermoelectric power generator, etc.

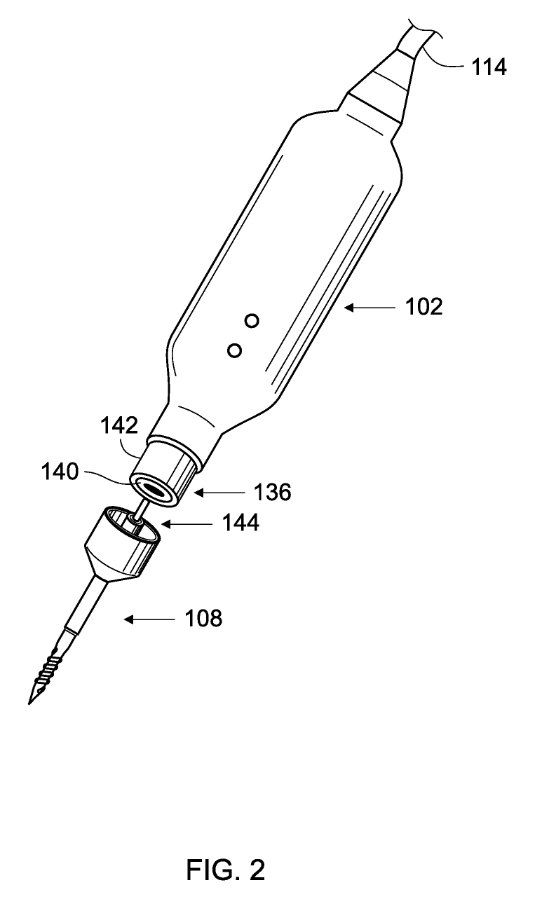

FIG. 2 shows a partial view of the myoablation system shown in FIG. 1. Handle 102 is operably connected by mix cable 114 to switch 116 (not shown). Thermal needle electrode tip 108 is showing detached from Handle 102. Handle 102 has a coaxial jack 136. Coaxial jack 136 has a central conductor 138 (not visible), a middle conductor 140, and an outer conductor 142. Thermal needle electrode tip 108 has a coaxial plug 144.

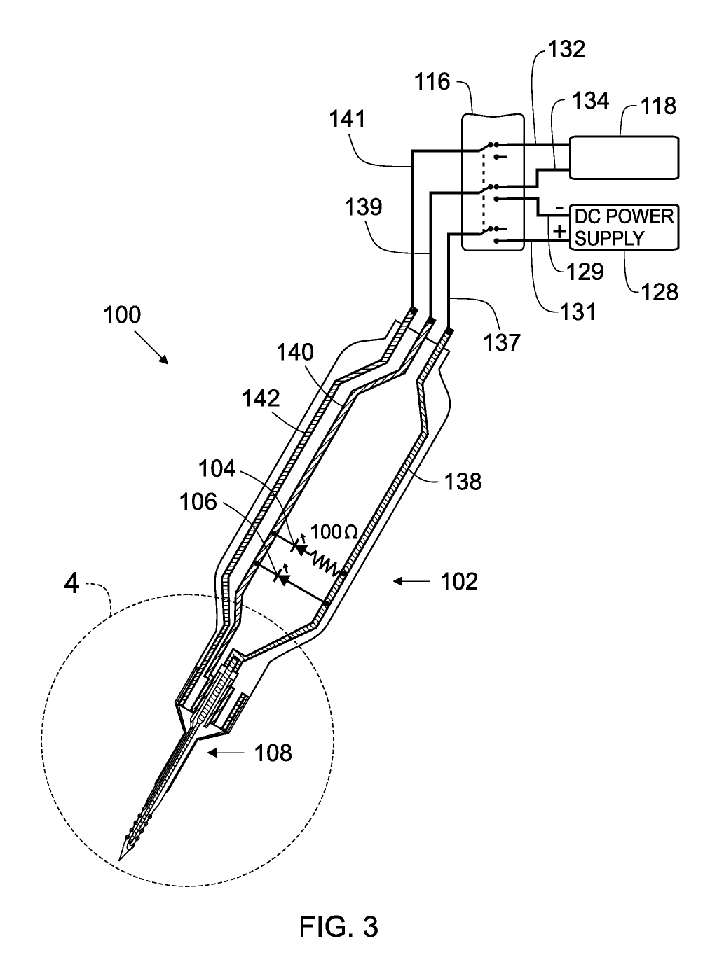

FIG. 3 shows a schematic view of the myoablation system shown in FIG. 1. Hand piece 100 comprises handle 102 and thermal needle electrode tip 108. Handle 102 is attached to thermal needle electrode tip 108.

Muscle detector 118 is connected by a sampling ground 132 wire, and by a sampling input 134 wire to switch 116 terminals. Input cable 126 (not shown) contains sampling ground 132 wire, and sampling input 134 wire.

Power supply 128 is connected by a negative wire 129, and by a positive wire 131 to switch 116 terminals. Power cable 130 (not shown) contains negative wire 129, and positive wire 131.

Switch 116 is connected by an energizer wire 137 to central conductor 138. Additionally, switch 116 is connected by a common wire 139 to middle conductor 140. Similarly, switch 116 is connected by a detector wire 141 to outer conductor 142. Mix cable 114 (not shown) contains energizer wire 137, and common wire 139, and detector wire 141.

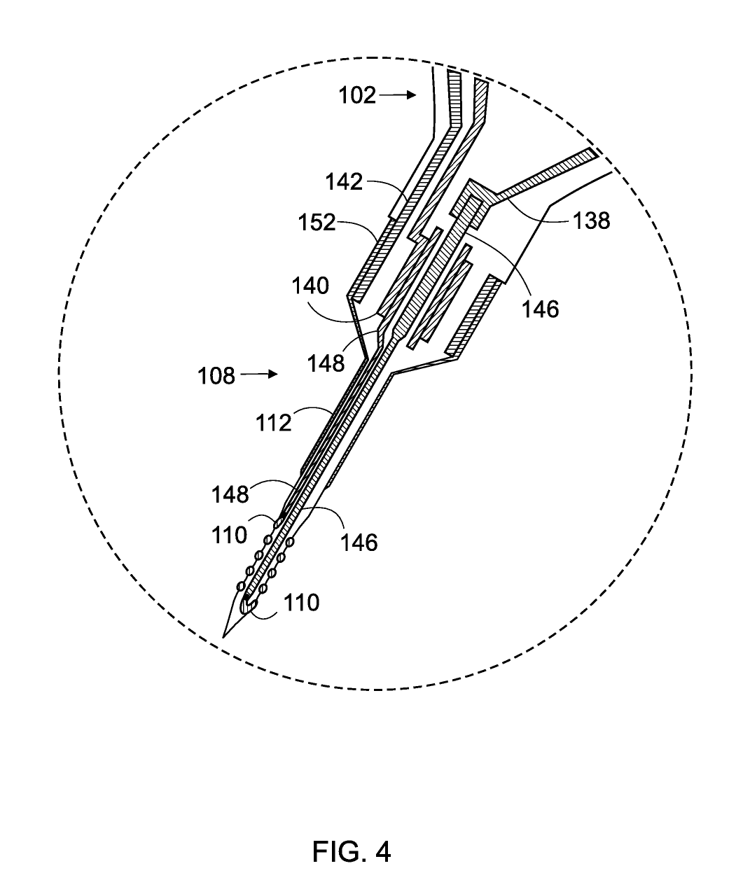

Energize indicator 104 is connected to central conductor 138, and to middle conductor 140. Likewise, time indicator 106 is connected to central conductor 138, and to middle conductor 140. Connection between handle 102 and thermal needle electrode tip 108 is shown enlarged in FIG. 4 for clarity.

I contemplate that negative wire 129, positive wire 131, sampling ground 132, sampling input 134, energizer wire 137, common wire 139, and detector wire 141 are made of shielded highly-conductive copper alloy available from Industrial Electric Wire & Cable, Inc. 5001 S. Towne Drive, New Berlin, Wis. 53151 (www.iewc.com), but others materials are also suitable.

I presently propose that central conductor 138, middle conductor 140, and outer conductor 142 are made of highly-conductive gold-plated copper, but others materials are also suitable.

FIG. 4 shows an enlarged partial view of the myoablation system shown in FIG. 3. Handle 102 is attached to thermal needle electrode tip 108. Central conductor 138 is connected by a central rod 146 to resistive heating element 110. Resistive heating element 110 is connected by a middle rod 148 to middle conductor 140. Outer conductor 142 is connected by an outer rod 152 to percutaneous return electrode 112.

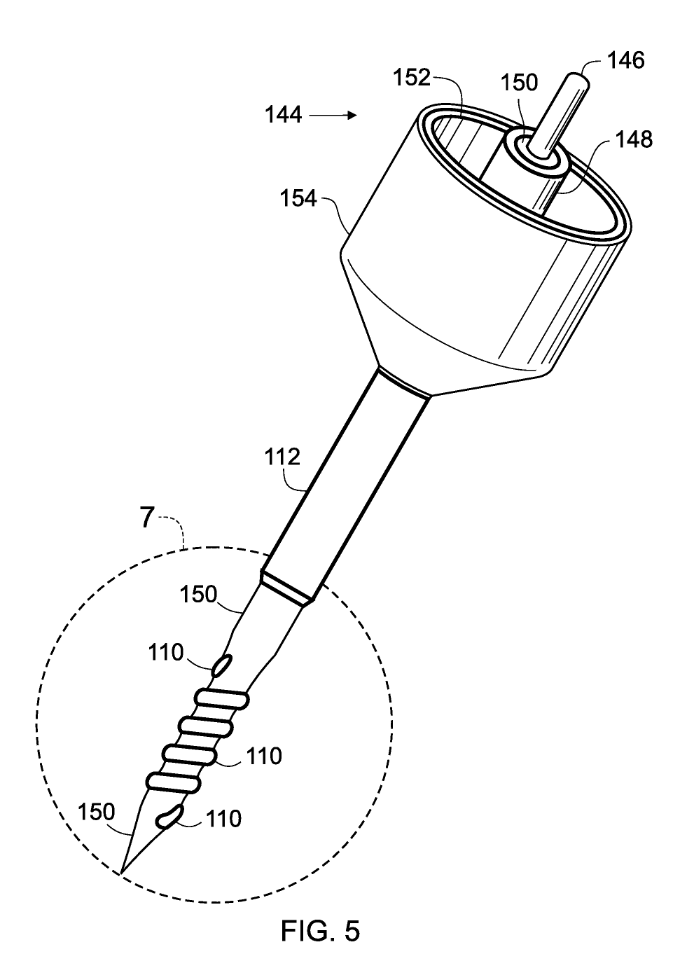

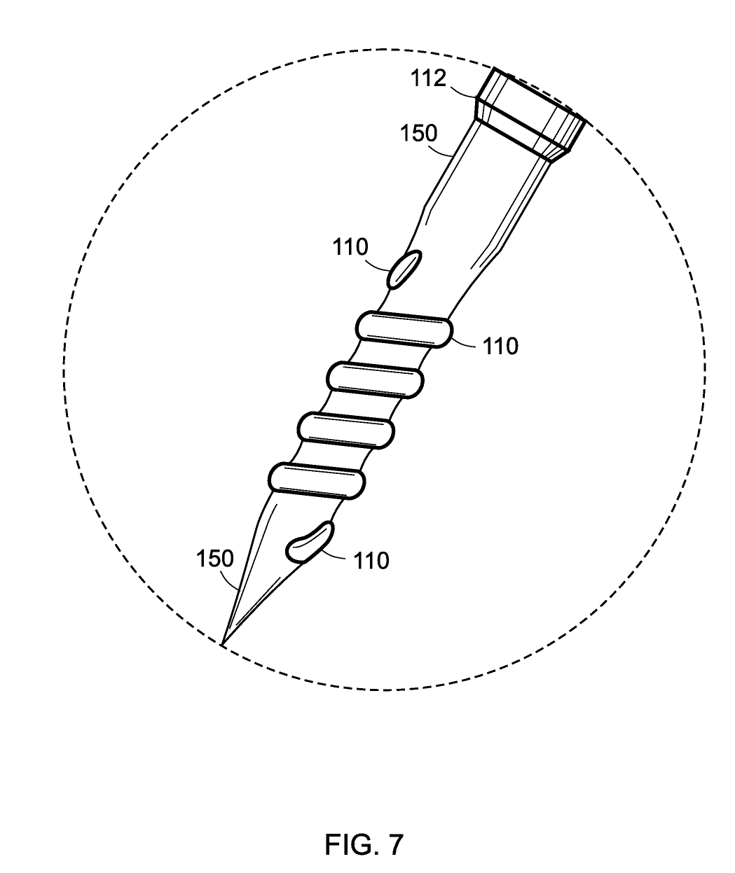

FIG. 5 shows a perspective view of thermal needle electrode tip 108 shown in FIG. 2. Thermal needle electrode tip 108 of FIG. 5 has coaxial plug 144, an electrothermal insulator matrix 150, an outer insulator 154, percutaneous return electrode 112, and resistive heating element 110. Coaxial plug 144 comprises central rod 146, electrothermal insulator matrix 150, middle rod 148, and outer rod 152. The distal end of thermal needle electrode tip 108 is shown enlarged in FIG. 7 for clarity.

I contemplate that percutaneous return electrode 112 have a circular cross section of 1 mm and be 10 mm long. Notwithstanding it can have different cross sections, such oval, triangular, rectangular etc., and different dimensions.

I presently propose that resistive heating element 110 have an overall resistance value of 5.2 ohms and be arranged in a 10 mm long coil of 33 loops. However it can have different resistant values, and different arrangements.

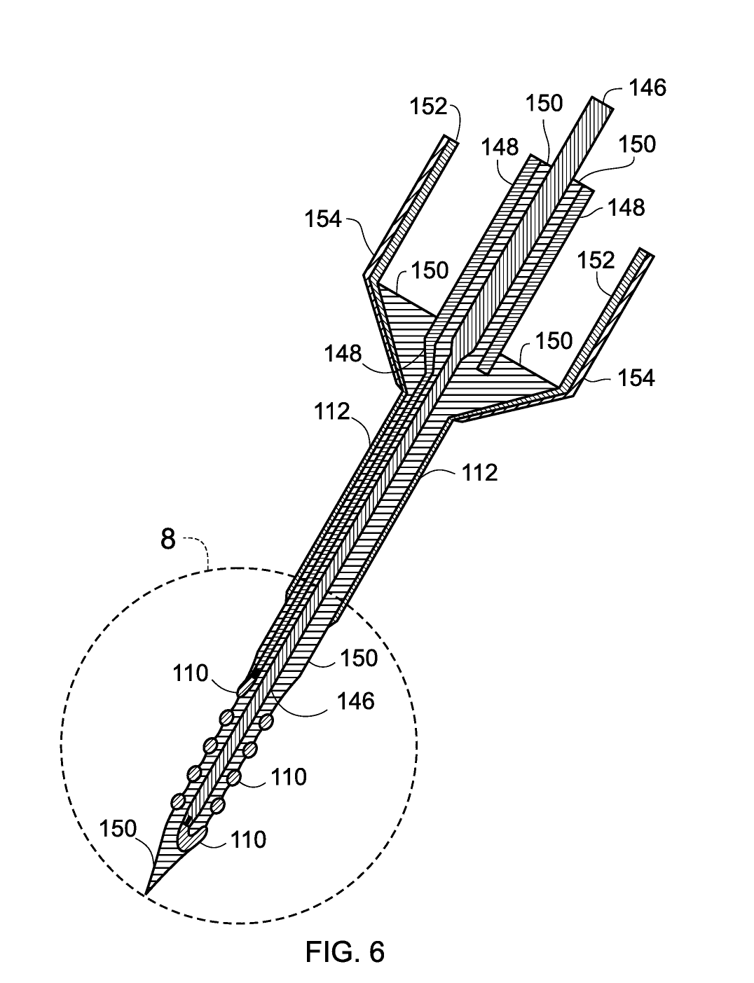

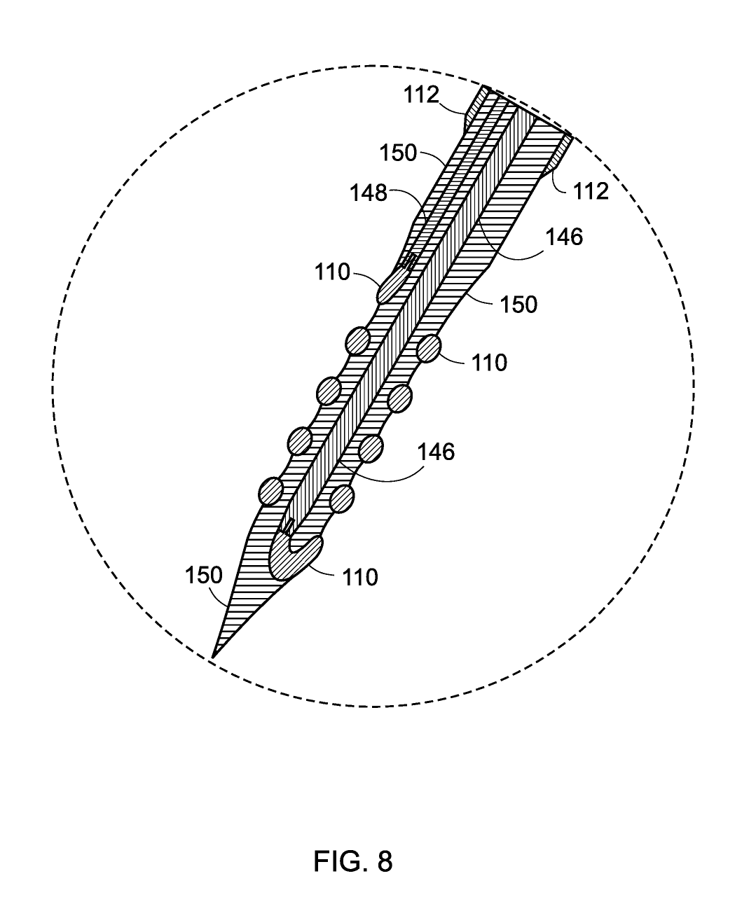

FIG. 6 shows a perspective sectional view of thermal needle electrode tip 108 shown in FIG. 5. Central rod 146 is connected to resistive heating element 110. Resistive heating element 110 is connected to middle rod 148. Outer rod 152 is connected to percutaneous return electrode 112. Central rod 146, middle rod 148, and resistive heating element 110 are embedded in electrothermal insulator matrix 150. Outer insulator 154 covers outer rod 152. The distal end of thermal needle electrode tip 108 is shown enlarged in FIG. 8 for clarity.

I presently contemplate that resistive heating element 110 is made of nickel-chromium resistance wire of 0.20 mm in diameter, available from WireTronic Inc. 19604 Mella Drive, Volcano, Calif. 95689-9786 USA (www.wiretron.com). However it can consist of any other material that can convert electrical energy into thermal energy.

I presently propose that central rod 146, middle rod 148, outer rod 152, and percutaneous return electrode 112 are made of stainless steel, but others materials are also suitable.

I currently contemplate that electrothermal insulator matrix 150, and outer insulator 154 are made of mullite available from McDanel Advanced Ceramic Technologies 510 9th Ave. Beaver Falls, Pa. 15010 (www.mcdanelceramics.com), but others materials are also suitable.

FIG. 7 shows an enlarged view of the distal end of thermal needle electrode tip 108 shown in FIG. 5. Exterior surface of resistive heating element 110 is not covered by electrothermal insulator matrix 150. Resistive heating element 110 is separated from percutaneous return electrode 112 by electrothermal insulator matrix 150.

FIG. 8 shows an enlarged sectional view of the distal end of thermal needle electrode tip 108 shown in FIG. 6. Central rod 146 is connected to resistive heating element 110. Resistive heating element 110 is connected to middle rod 148. Central rod 146, middle rod 148, and resistive heating element 110 are embedded in electrothermal insulator matrix 150. Exterior surface of resistive heating element 110 is not covered by electrothermal insulator matrix 150. Central rod 146, middle rod 148, and resistive heating element 110 are separated from percutaneous return electrode 112 by electrothermal insulator matrix 150.

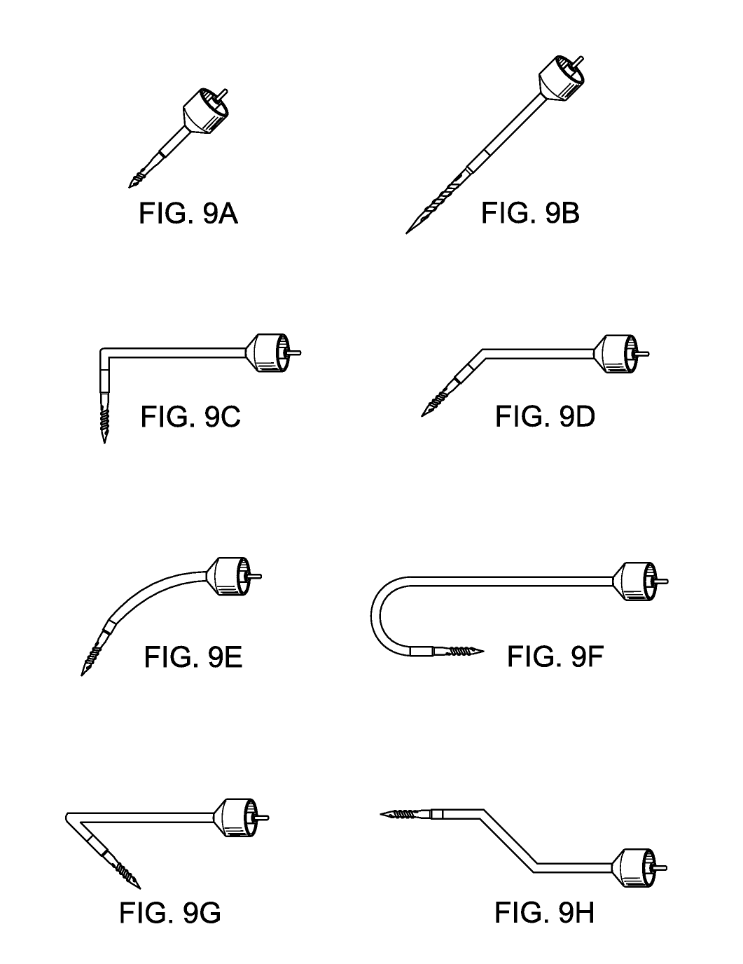

FIGS. 9A, 9B, 9C, 9D, 9E, 9F, 9G, and 9H shown additional embodiments of thermal needle electrode tip 108, but others embodiments are also possible.

FIG. 10 shows a schematic view of circuitry of muscle detector 118 shown in FIG. 1. Muscle detector 118 circuitry of FIG. 10 comprises graphic display 120, gain control 122, and sweep time control 124. Muscle detector 118 circuitry is connected to sampling ground 132 wire and to sampling input 134 wire.

I currently contemplate that muscle detector 118 comprises components available from National Semiconductor Corporation, 2900 Semiconductor Drive, Santa Clara, Calif. 95051 (www.national.com), but others components are also suitable.

I presently propose that graphic display 120 comprises LEDs. Notwithstanding it can comprise liquid crystal display (LCD), cathode-ray tube (CRT) display, plasma display panel (PDP), organic light emitting diode (OLED), etc.

I presently contemplate that gain control 122, and sweep time control 124 are carbon potentiometers available from State Electronics 36 Route 10 East Hanover, N.J. 07936 (www.potentiometer.com), but others components are also suitable.

Muscle detector 118 repeatedly draws a bar or horizontal line called the trace across graphic display 120 (screen) from left to right. One of the controls, sweep time control 124 (horizontal control), regulates the NE555P timer frequency, and sets the speed at which the line is drawn. Another control, gain control 122 (vertical control), sets the degree to which the LM324 amplifier magnifies the low-level sampling input 134 signal. The resulting trace is a plot of voltage against time. Additionally, part of LM324 amplifier is configured as a 60 Hz notch filter to minimize interference.

When gain control 122 potentiometer is set at 10 megaohms, the resulting gain ratio is 1:1011. When gain control 122 potentiometer is set at 0 ohms, the resulting gain ratio is 1:11.

Gain control 122 potentiometer knob is arranged in such a way that a full clockwise turn results in a gain ratio of 1:1011 (high gain). Gain control 122 potentiometer knob is arranged in such a way that a full counterclockwise turn results in a gain ratio of 1:11 (low gain).

When sweep time control 124 potentiometer is set at 0 ohms, the resulting sweep frequency is 500 Hz. When sweep time control 124 potentiometer is set at 1 megaohm, the resulting sweep frequency is 0.6 Hz.

Sweep time control 124 potentiometer knob is arranged in such a way that a full clockwise turn results in a sweep frequency of 500 Hz (high speed). Sweep time control 124 potentiometer knob is arranged in such a way that a full counterclockwise turn results in a sweep frequency of 0.6 Hz (low speed).

### Operation

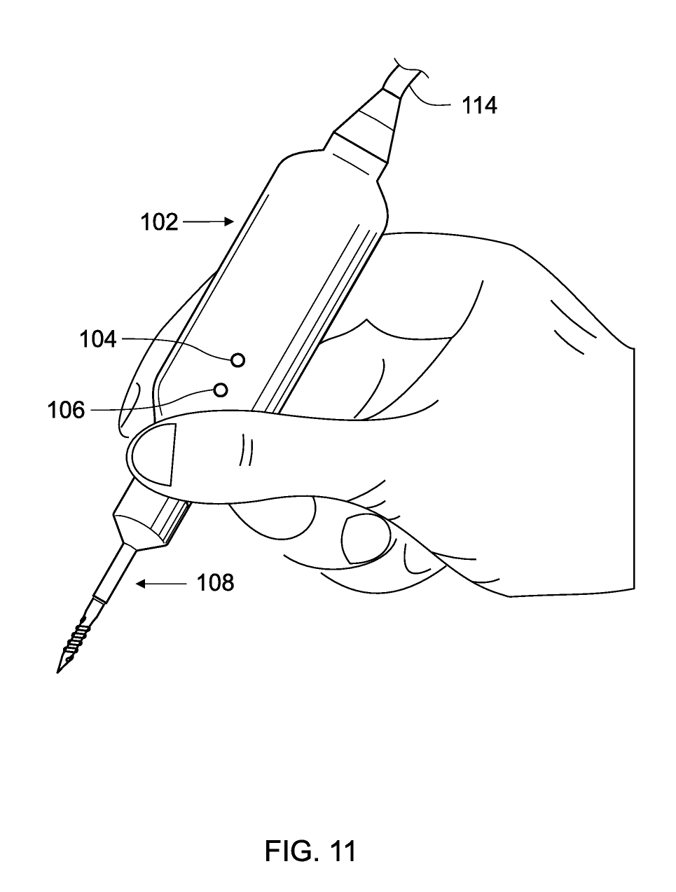

### First Embodiment—FIGS. 11, 12A, 12B, 13A, 13B, 14, 15, 16, 17A. 17B

FIG. 11 shows an operational view of the myoablation system shown in FIG. 1. Hand piece 100 shown in FIG. 11 is hold like a pencil. Handle 102 in connected by mix cable 114 to switch 116 (not shown). Energize indicator 104 green LED lights when thermal needle electrode tip 108 is being energized. Time indicator 106 yellow LED blinks (at a 2 Hz blinking rate) when thermal needle electrode tip 108 is being energized.

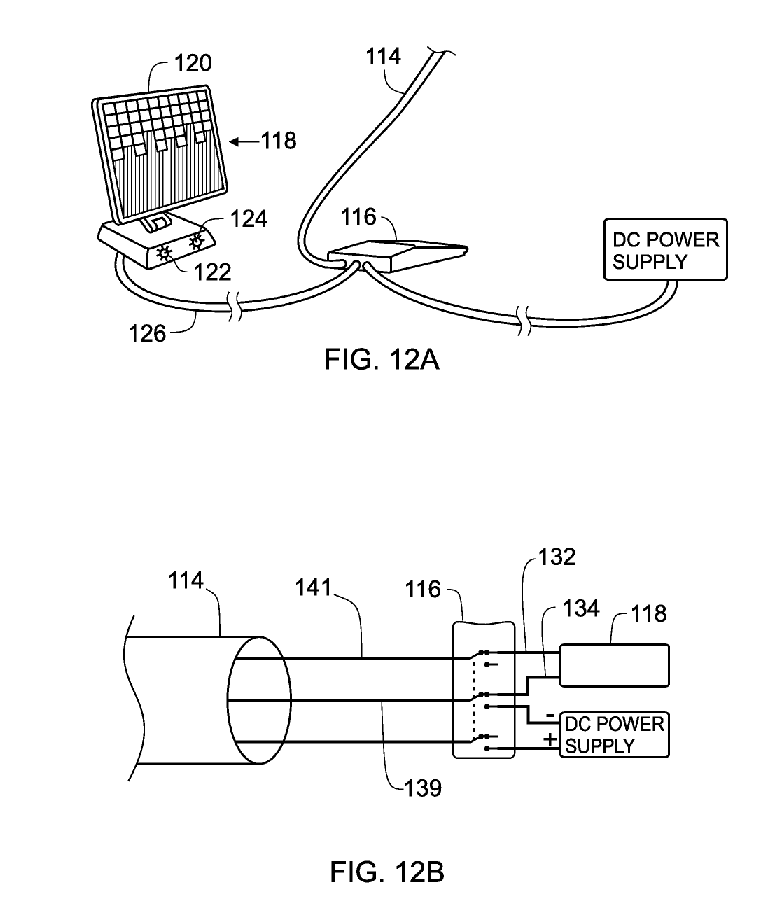

FIGS. 12A and 12B shown an operational view of the myoablation system shown in FIG. 1. When switch 116 is undisturbed, thermal needle electrode tip 108 (shown in FIG. 11) is operably connected by mix cable 114 and input cable 126 to muscle detector 118. In this circuit configuration the signal coming from thermal needle electrode tip 108 (shown in FIG. 11) travels by detector wire 141, and common wire 139 (contained in mix cable 114) to switch 116; then this signal travels from switch 116 by sampling ground 132 wire, and sampling input 134 wire (contained in input cable 126) to muscle detector 118. This signal is plotted in graphic display 120 of muscle detector 118. Muscle detector 118 repeatedly draws this signal across graphic display 120 from left to right. Gain control 122 (vertical control), sets the size at which this signal is presented in graphic display 120. Sweep time control 124 (horizontal control), sets the speed at which this signal is presented in graphic display 120.

Gain control 122 potentiometer knob is arranged in such a way that a full clockwise turn results in a maximum size at which this signal is presented in graphic display 120 (gain ratio 1:1011). Gain control 122 potentiometer knob is arranged in such a way that a full counterclockwise turn results in a minimum size at which this signal is presented in graphic display 120 (gain ratio 1:11).

Sweep time control 124 potentiometer knob is arranged in such a way that a full clockwise turn results in a maximum speed at which this signal is presented in graphic display 120 (sweep frequency of 500 Hz). Sweep time control 124 potentiometer knob is arranged in such a way that a full counterclockwise turn results in a minimum speed at which this signal is presented in graphic display 120 (sweep frequency of 0.6 Hz).

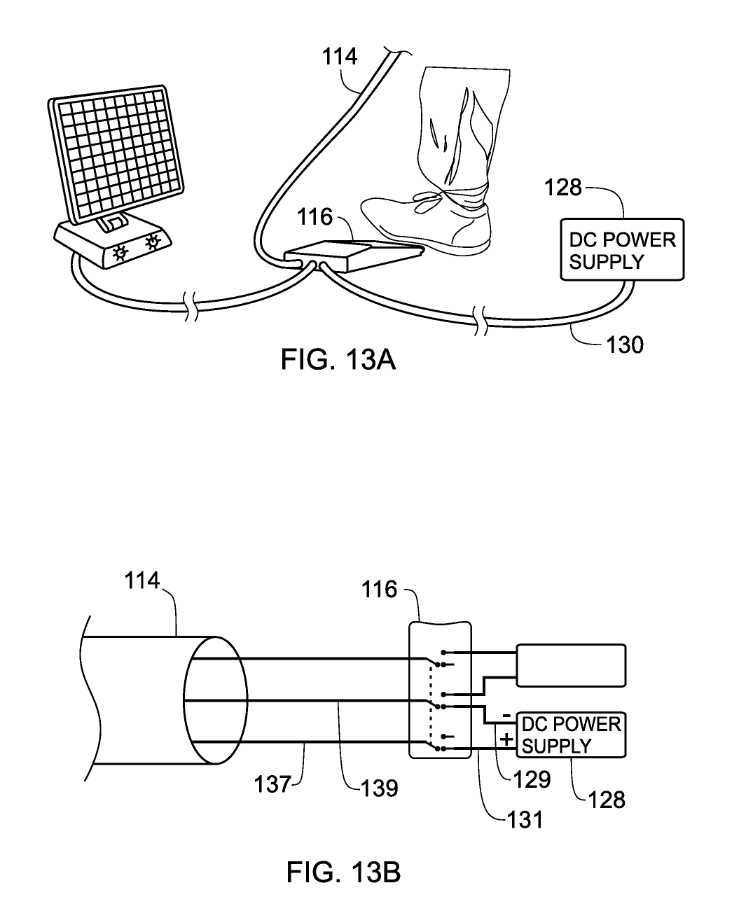

FIGS. 13A and 13B shown an operational view of the myoablation system shown in FIG. 1. When switch 116 is pressed, thermal needle electrode tip 108 (shown in FIG. 11) is operably connected by mix cable 114 and power cable 130 to power supply 128. In this circuit configuration the electrical energy coming from power supply 128 travels by positive wire 131, and negative wire 129 (contained in power cable 130) to switch 116; then this electrical energy travels from switch 116 by energizer wire 137, and common wire 139 (contained in mix cable 114) to thermal needle electrode tip 108 (shown in FIG. 11). At the same time, energize indicator 104 green LED and time indicator 106 yellow LED (shown in FIG. 11) light. Time indicator 106 yellow LED blinks at a 2 Hz blinking rate, allowing the physician to calculate the time thermal needle electrode tip 108 remains energized.

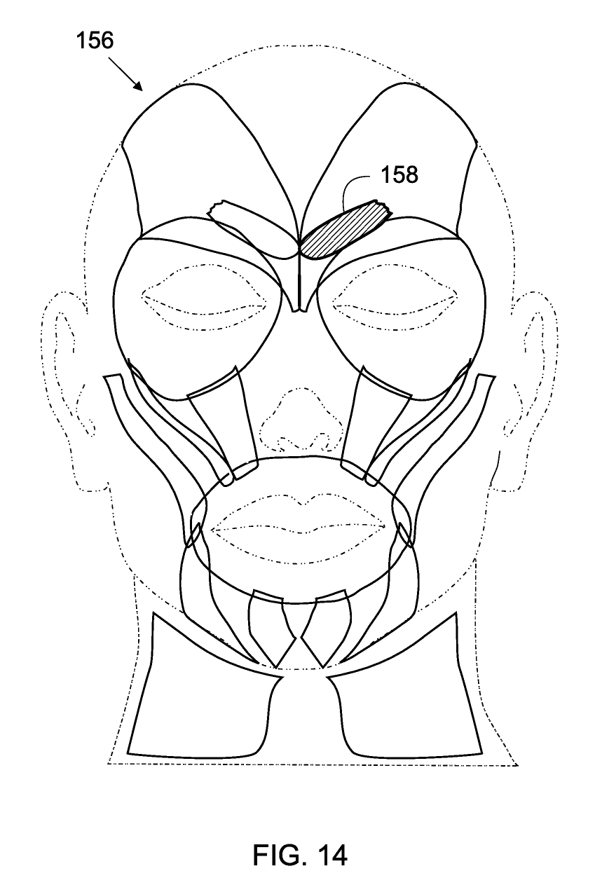

FIG. 14 shows a schematic view of the facial muscles 156 anatomy. Facial muscles 156 are subcutaneous (just under the skin) muscles that control facial expression. They generally originate on bone, and insert on the skin of the face.

Facial muscles 156 are generally shown in FIG. 14, and are not individually described for simplicity. The facial muscles 156 comprise: occipitofrontalis, procerus, nasalis muscle, depressor septi nasi, orbicularis oculi, corrugator supercilii, depressor supercilii, auricular muscles (anterior, superior, posterior), orbicularis oris, depressor anguli oris, risorius, zygomaticus major, zygomaticus minor, levator labii superioris, levator labii superioris alaeque nasi, depressor labii inferioris, levator anguli oris, buccinator, mentalis, and platysma. The platysma muscle is innervated by the facial nerve. Although it is mostly in the neck, due to its common innervation it can sometimes also be considered a muscle of facial expression.

The myoablation system can be used to perform a myoablation procedure on one or more facial muscles 156. Nevertheless the myoablation system can be used for performing a myoablation procedure in other muscles. The left side corrugator supercilii muscle 158 is selected to illustrate how to use the myoablation system to perform a myoablation procedure. The goal of the following myoablation procedure is to reduce left side corrugator supercilii muscle 158 activity.

The myoablation procedure is accomplished in three steps as follows:

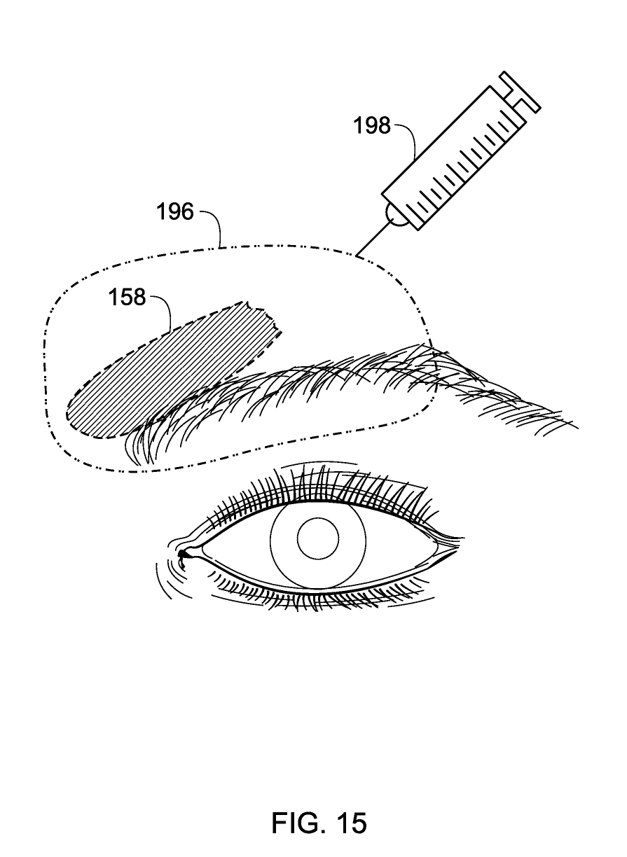

(1) Local anesthesia procedure (FIG. 15)

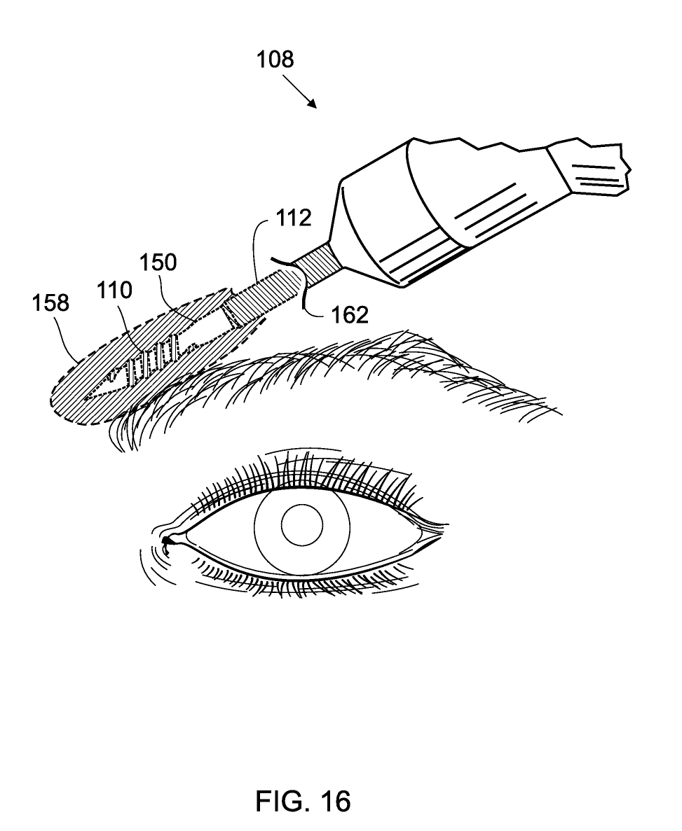

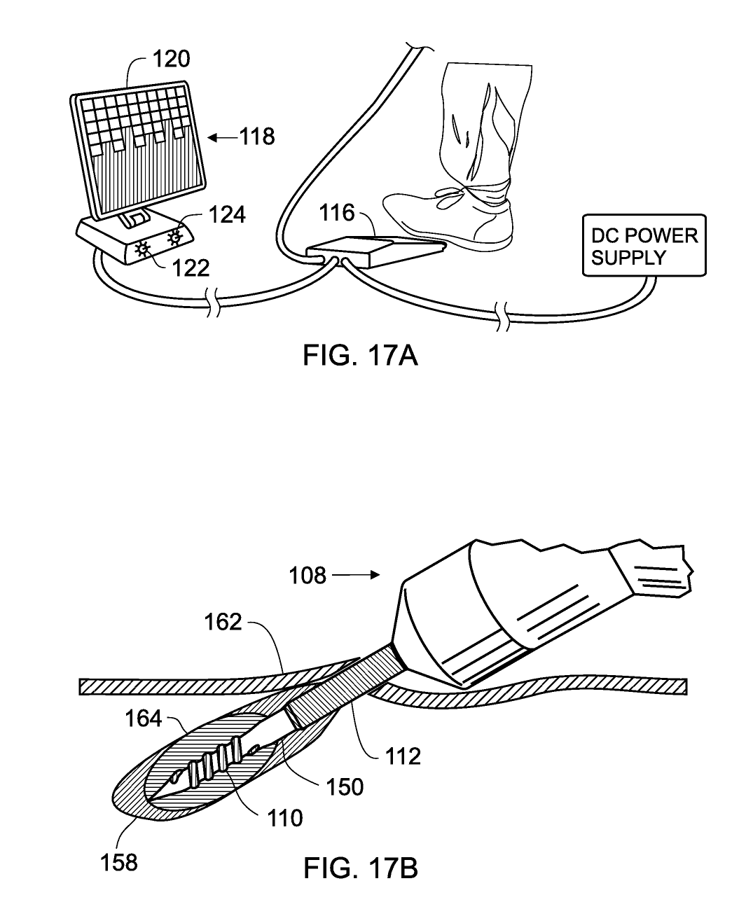

(2) Percutaneous muscle detection (FIGS. 16 and 17A)

(3) Increasing the internal energy in the muscle detected (FIGS. 17A and 17B)

FIG. 15 shows a schematic view of a local anesthesia procedure. Corrugator supercilii muscles are paired muscles that control frowning. The patient is asked to frown, and the subcutaneous anatomical location of left side corrugator supercilii muscle 158 is marked with ink. The skin and subcutaneous tissues of an operative area 196 are infiltrated with a syringe 198 containing a local anesthetic solution. The composition of this local anesthetic solution is as follows: 15.00 cc of normal saline solution (0.9% NaCl), 2.00 cc of 2% lidocaine, and 0.25 mg of epinephrine. Notwithstanding other local anesthetic solutions are also suitable.

FIG. 16 shows a schematic view of a myoablation procedure. Thermal needle electrode tip 108 is inserted percutaneously and directed toward left side corrugator supercilii muscle 158 in such a way that resistive heating element 110 is in left side corrugator supercilii muscle 158, and percutaneous return electrode 112 is in contact with skin 162. Resistive heating element 110 pickups left side corrugator supercilii muscle 158 electrical signal. Percutaneous return electrode 112 provides an electrical return path. Electrothermal insulator matrix 150 provides electrical and thermal insulation.

FIGS. 17A and 17B shows a schematic view of a myoablation procedure. The trace in graphic display 120 of muscle detector 118 indicates that resistive heating element 110 of thermal needle electrode tip 108 is in left side corrugator supercilii muscle 158. Then switch 116 is pressed and heat is delivered through resistive heating element 110 in left side corrugator supercilii muscle 158. Heat increases the internal energy of muscle tissue resulting in a well-demarcated area of defunctionalized muscle (corrugator supercilii muscle ablated area 164). Next, the patient is asked to frown to determine the degree of reduction of left side corrugator supercilii muscle 158 activity. The procedure is repeated until the desire effect is achieved. In most cases 3 sets of 20 seconds in duration each suffice to achieve the desired degree of reduction of left side corrugator supercilii muscle 158 activity.

Since typical voltage of an intact left side corrugator supercilii muscle 158 is 5.2 millivolts, gain control 122 knob is set at maximum gain (full clockwise turn, gain ratio 1:1011). Sweep time control 124 knob is set at maximum speed (full clockwise turn, sweep frequency of 500 Hz).

Electrical energy supplied to resistive heating element 110 is 4.5 volts DC, 2 Amps. This current increases resistive heating element 110 temperature to 98.0° C. Left side corrugator supercilii muscle 158 in contact with resistive heating element 110 increases its temperature up to 85.0° C. This produces a disruption of the muscle contractile proteins actin and myosin, creating an area of defunctionalized muscle (corrugator supercilii muscle ablated area 164).

Electrothermal insulator matrix 150 limits the conduction of heat generated by resistive heating element 110. The temperature of percutaneous return electrode 112 in contact with skin 162 is 37.5° C., therefore skin 162 is undamaged.

The dimensions of defunctionalized muscle (corrugator supercilii muscle ablated area 164) depend on how many seconds resistive heating element 110 is in contact with muscle. When resistive heating element 110 is in contact with left side corrugator supercilii muscle 158 for 20 seconds, the dimensions of corrugator supercilii muscle ablated area 164 are 16.0×3.0×3.0 mm. When resistive heating element 110 is in contact with left side corrugator supercilii muscle 158 for 30 seconds, the dimensions of corrugator supercilii muscle ablated area 164 are 17.4×3.7×3.7 mm.

### Description

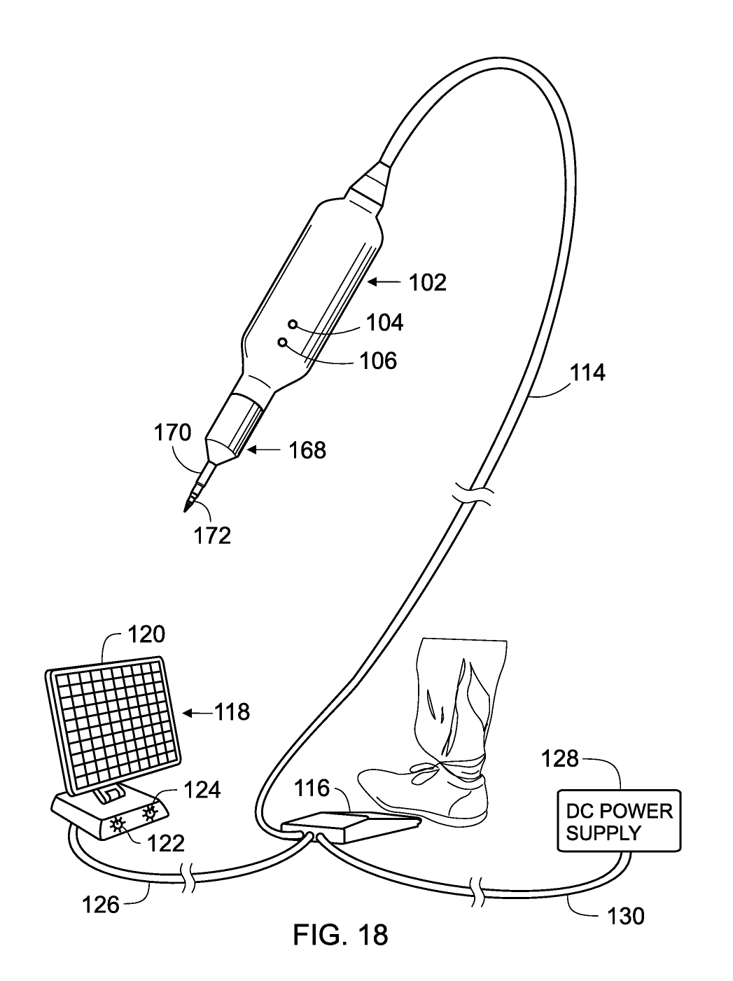

### Second Embodiment—FIGS. 18, 19, 20, 21, 22, 23, 24, 25, 26, 27A, 27B, 27C, 27D, 27E, 27F, 27G, 27H

FIG. 18 shows an overall view of the myoablation system of the second embodiment. The myoablation system of FIG. 18 has handle 102 and a detachable radio frequency energy needle electrode tip 168. Handle 102 has energize indicator 104 and time indicator 106. Radio frequency energy needle electrode tip 168 has a coaxial return electrode 170 and an accessory conductor 172.

Handle 102 is operably connected by mix cable 114 to switch 116. Switch 116 is operably connected by input cable 126 to muscle detector 118. Muscle detector 118 has graphic display 120, gain control 122, and sweep time control 124. In addition, switch 116 is operably connected by power cable 130 to power supply 128.

I contemplate that muscle detector 118 of this embodiment has its own power source (not shown).

I presently propose that power supply 128 of this embodiment consists of a 2 Amps regulated DC power source with a variable output range of 4.5 to 9 volt DC. Nevertheless other power sources are also suitable, such as a battery, electrochemical cell, thermoelectric power generator, etc.

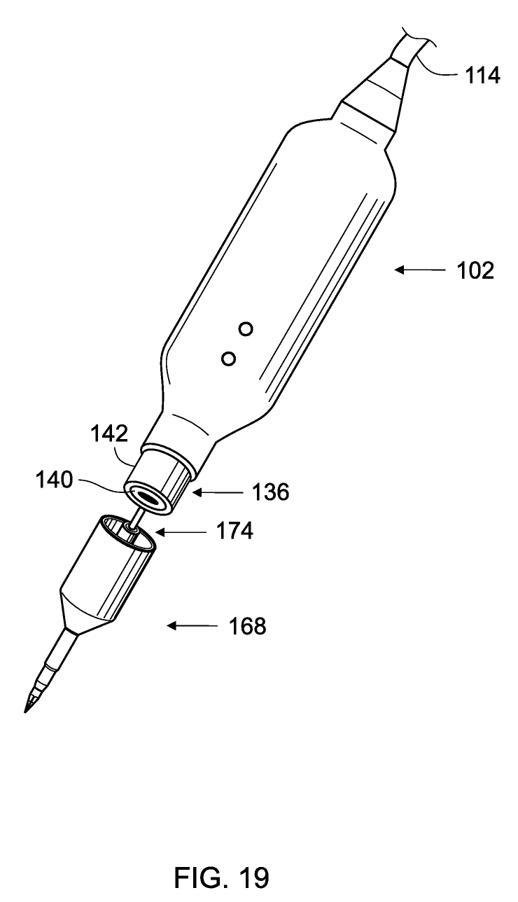

FIG. 19 shows a partial view of the myoablation system shown in FIG. 18. Handle 102 is operably connected by mix cable 114 to switch 116 (not shown). Radio frequency energy needle electrode tip 168 is showing detached from Handle 102. Handle 102 has coaxial jack 136. Coaxial jack 136 has central conductor 138 (not visible), middle conductor 140, and outer conductor 142. Radio frequency energy needle electrode tip 168 has a coaxial connector 174.

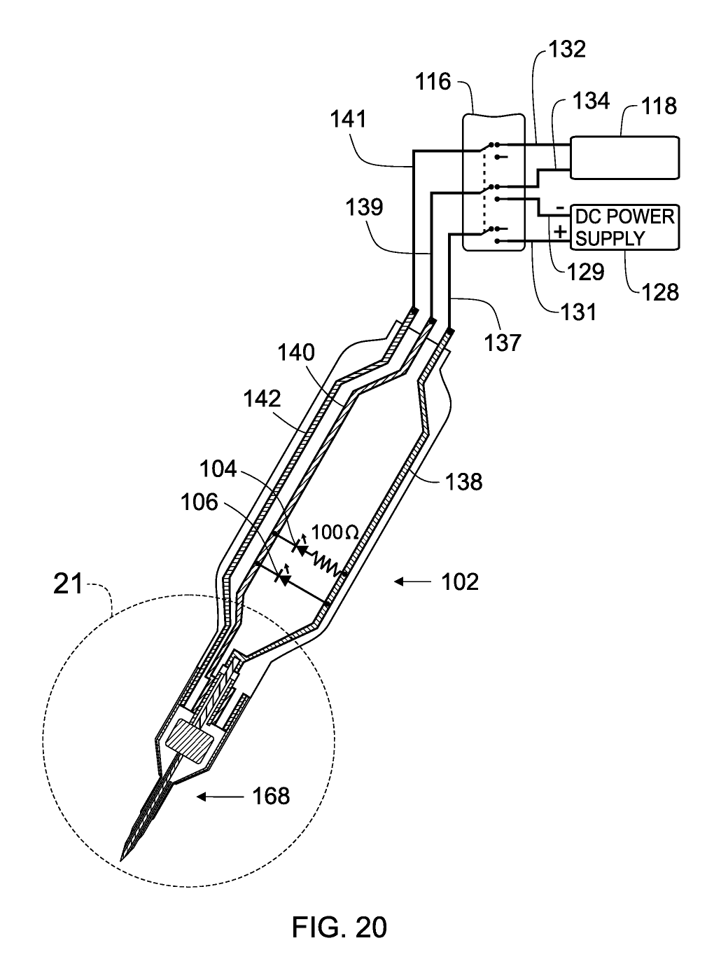

FIG. 20 shows a schematic view of the myoablation system shown in FIG. 18. Handle 102 is attached to radio frequency energy needle electrode tip 168.

Muscle detector 118 is connected by sampling ground 132 wire, and by sampling input 134 wire to switch 116 terminals. Input cable 126 (not shown) contains sampling ground 132 wire, and sampling input 134 wire.

Power supply 128 is connected by negative wire 129, and by positive wire 131 to switch 116 terminals. Power cable 130 (not shown) contains negative wire 129, and positive wire 131.

Switch 116 is connected by energizer wire 137 to central conductor 138. Additionally, switch 116 is connected by common wire 139 to middle conductor 140. Similarly, switch 116 is connected by detector wire 141 to outer conductor 142. Mix cable 114 (not shown) contains energizer wire 137, and common wire 139, and detector wire 141.

Energize indicator 104 is connected to central conductor 138, and to middle conductor 140. Likewise, time indicator 106 is connected to central conductor 138, and to middle conductor 140.

When switch 116 is undisturbed, the signal coming from radio frequency energy needle electrode tip 168 travels by detector wire 141, and common wire 139 to switch 116; then this signal travels from switch 116 by sampling ground 132 wire, and sampling input 134 wire to muscle detector 118.

When switch 116 is pressed, the electrical energy coming from power supply 128 travels by positive wire 131, and negative wire 129 to switch 116; then this electrical energy travels from switch 116 by energizer wire 137, and common wire 139 to radio frequency energy needle electrode tip 168. At the same time, energize indicator 104 green LED and time indicator 106 yellow LED light. Time indicator 106 yellow LED blinks at a 2 Hz blinking rate, allowing the physician to calculate the time radio frequency energy needle electrode tip 168 remains energized.

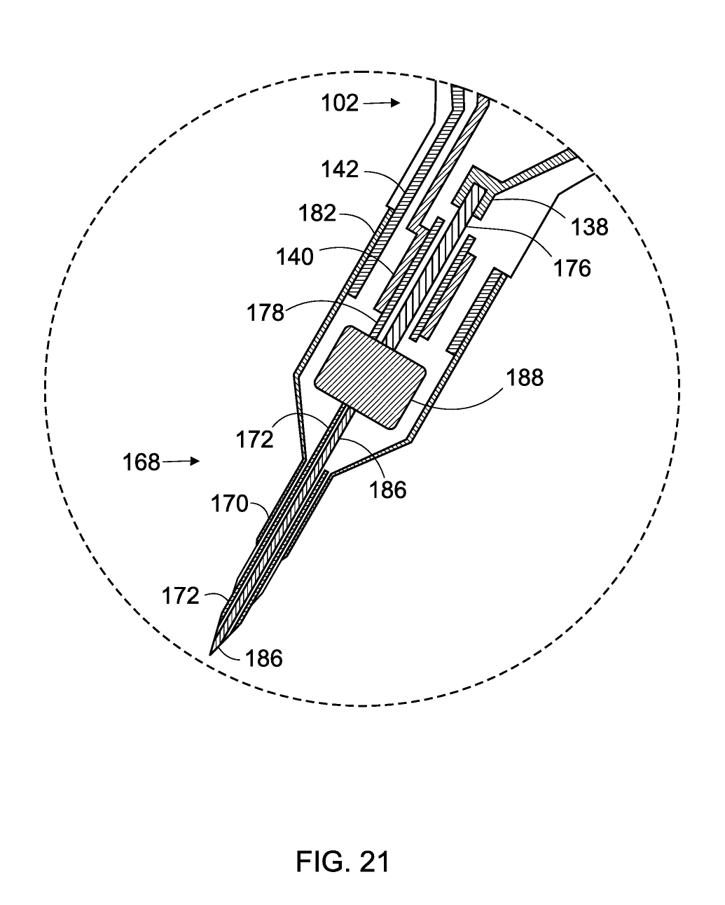

Connection between handle 102 and radio frequency energy needle electrode tip 168 is shown enlarged in FIG. 21 for clarity.

FIG. 21 shows an enlarged partial view of the myoablation system shown in FIG. 20. Handle 102 is attached to radio frequency energy needle electrode tip 168. Central conductor 138 is connected by a central coaxial conductor 176 to a radio frequency energy generator 188. Radio frequency energy generator 188 is connected by a middle coaxial conductor 178 to middle conductor 140. Outer conductor 142 is connected by an outer coaxial conductor 182 to coaxial return electrode 170.

Radio frequency energy generator 188 is connected to accessory conductor 172. In addition, radio frequency energy generator 188 is connected to a main conductor 186.

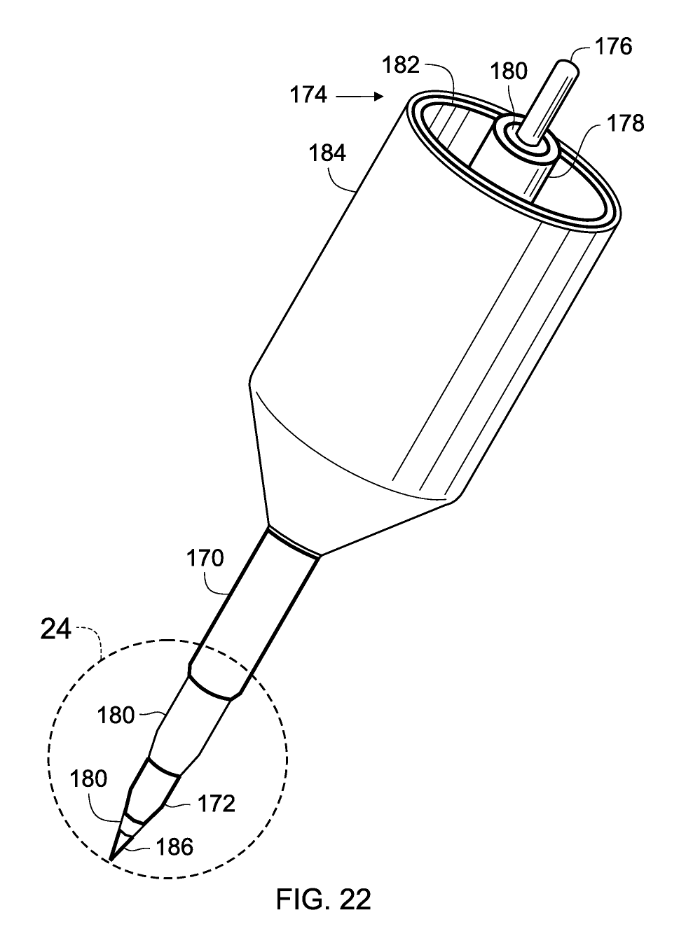

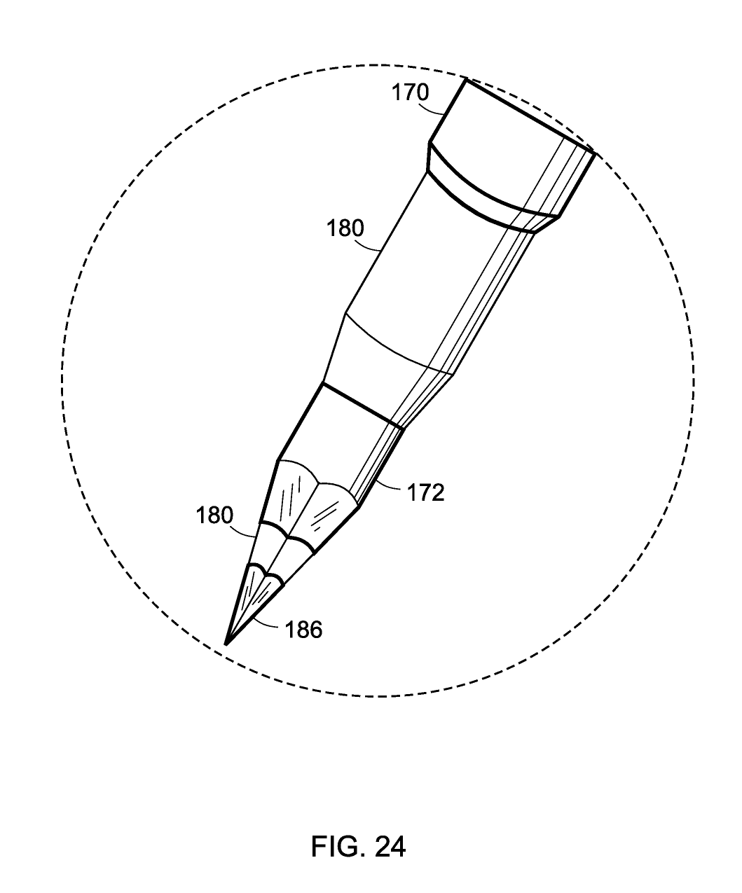

FIG. 22 shows a perspective view of radio frequency energy needle electrode tip 168 shown in FIG. 19. Radio frequency energy needle electrode tip 168 of FIG. 22 has coaxial connector 174, an insulator 180, an outer insulation 184, coaxial return electrode 170, accessory conductor 172, and main conductor 186. Coaxial connector 174 comprises central coaxial conductor 176, insulator 180, middle coaxial conductor 178, and outer coaxial conductor 182. The distal end of radio frequency energy needle electrode tip 168 is shown enlarged in FIG. 24 for clarity.

I contemplate that coaxial return electrode 170 have a circular cross section of 1 mm and be 10 mm long. Notwithstanding it can have different cross sections, such oval, triangular, rectangular etc., and different dimensions.

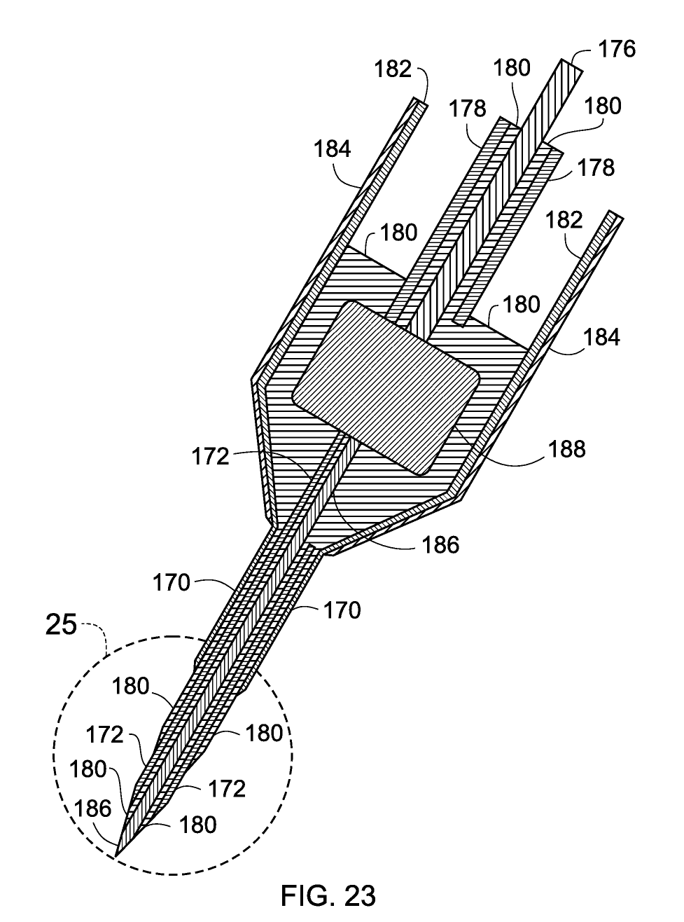

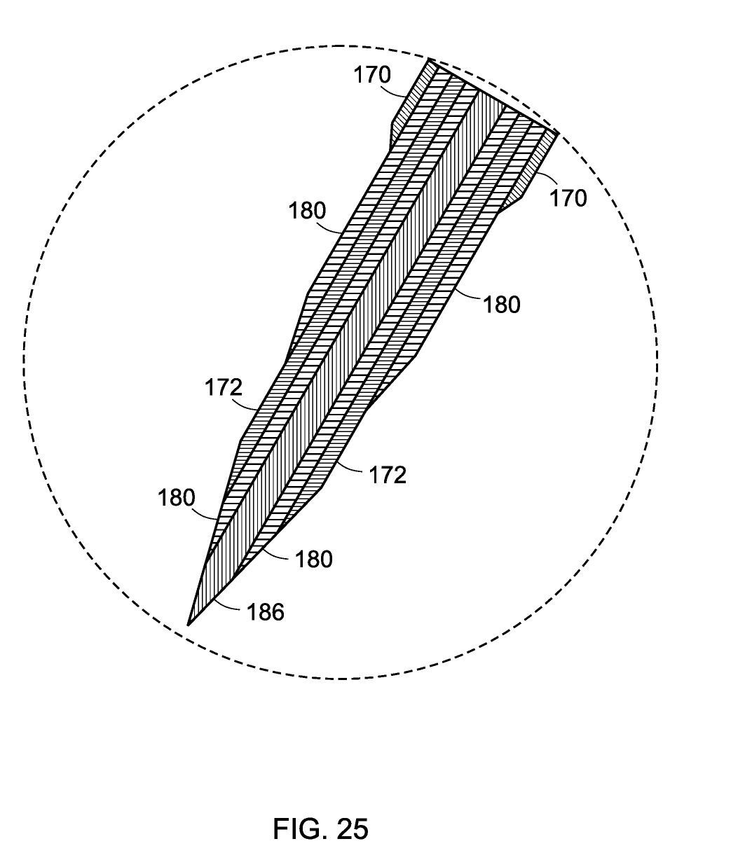

FIG. 23 shows a perspective sectional view of radio frequency energy needle electrode tip 168 shown in FIG. 22. Central coaxial conductor 176 is connected to radio frequency energy generator 188. Middle coaxial conductor 178 is connected to radio frequency energy generator 188. Outer coaxial conductor 182 is connected to coaxial return electrode 170. Radio frequency energy generator 188 is connected to accessory conductor 172. In addition, radio frequency energy generator 188 is connected to main conductor 186. Central coaxial conductor 176, middle coaxial conductor 178, radio frequency energy generator 188, accessory conductor 172, and main conductor 186 are embedded in insulator 180. Outer insulation 184 covers outer coaxial conductor 182. The distal end of thermal needle electrode tip 108 is shown enlarged in FIG. 25 for clarity.

I presently propose that central coaxial conductor 176, middle coaxial conductor 178, outer coaxial conductor 182, coaxial return electrode 170, accessory conductor 172, and main conductor 186 are made of stainless steel. Notwithstanding others materials are also suitable.

I currently contemplate that insulator 180, and outer insulation 184 are made of mullite available from McDanel Advanced Ceramic Technologies 510 9th Ave. Beaver Falls, Pa. 15010 (www.mcdanelceramics.com). Nevertheless others materials are also suitable.

FIG. 24 shows an enlarged view of the distal end of radio frequency energy needle electrode tip 168 shown in FIG. 22. Distal ends of both main conductor 186 and accessory conductor 172 are not covered by insulator 180. Coaxial return electrode 170, accessory conductor 172, and main conductor 186 are separated from each other by insulator 180.

FIG. 25 shows an enlarged sectional view of the distal end of radio frequency energy needle electrode tip 168 shown in FIG. 23. Distal ends of both main conductor 186 and accessory conductor 172 are not covered by insulator 180. Coaxial return electrode 170, accessory conductor 172, and main conductor 186 are separated from each other by insulator 180.

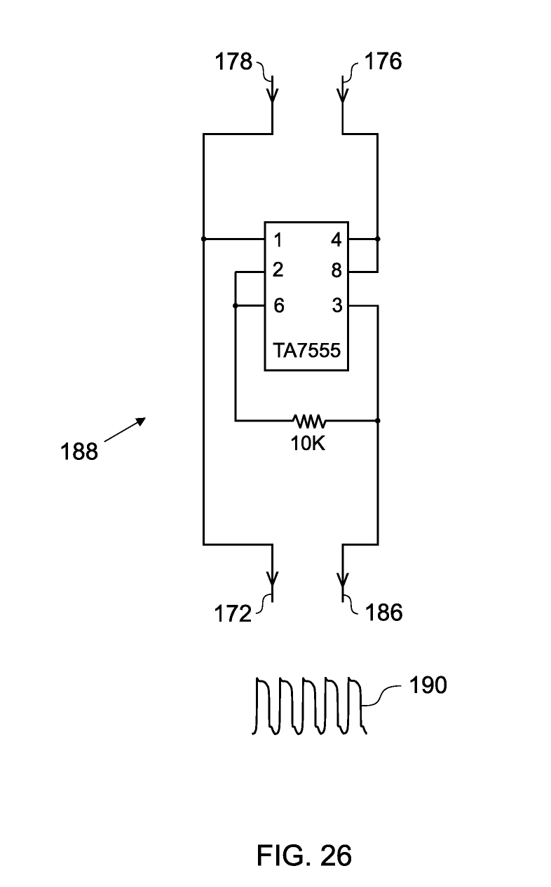

FIG. 26 shows a schematic view of a circuitry of radio frequency energy generator 188 shown in FIG. 21. Radio frequency energy generator 188 circuitry is powered by both middle coaxial conductor 178 and central coaxial conductor 176. Output of radio frequency energy generator 188 is delivered by both accessory conductor 172 and main conductor 186. An output wave form 190 of radio frequency energy generator 188 is shown.

I currently contemplate that radio frequency energy generator 188 comprises components available from Toshiba America Electronic Components, Inc. (TAEC) Headquarters: Irvine, Calif. (www.toshiba.com), but others components are also suitable.

I contemplate that when radio frequency energy generator 188 is powered with 9 volts DC, the typical values of output wave form 190 are frequency of 2.5 MHz, and a peak-to-peak voltage of 8.4 volts. Notwithstanding others values are also suitable.

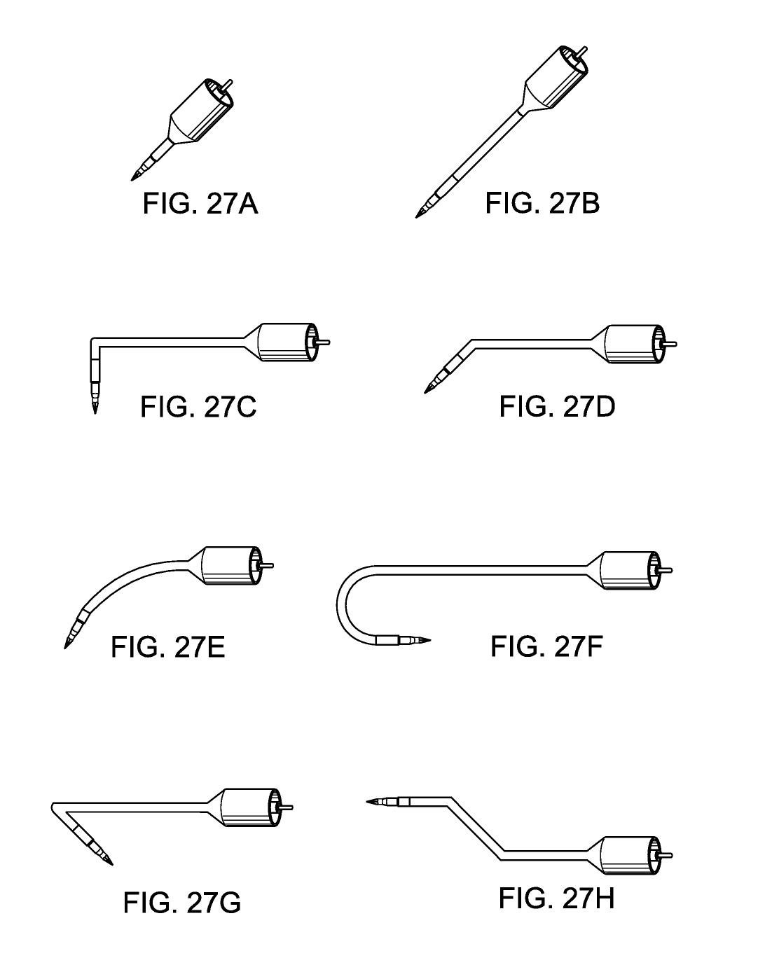

FIGS. 27A, 27B, 27C, 27D, 27E, 27F, 27G, and 27H shown additional embodiments of radio frequency energy needle electrode tip 168, but others embodiments are also possible.

### Operation

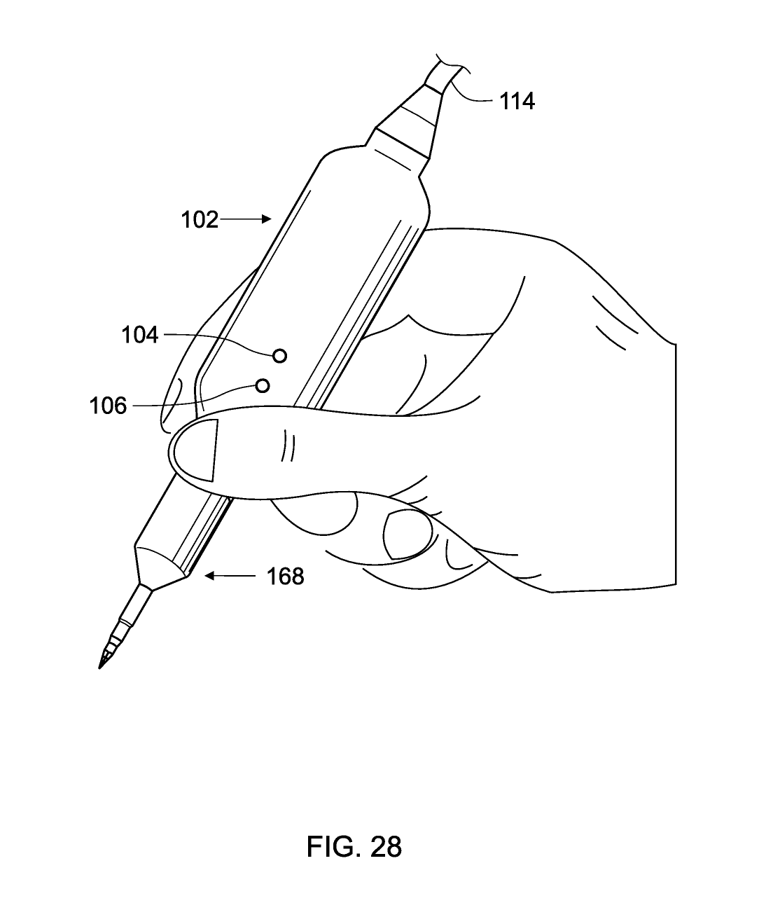

### Second Embodiment—FIGS. 28, 29, 30A, 30B

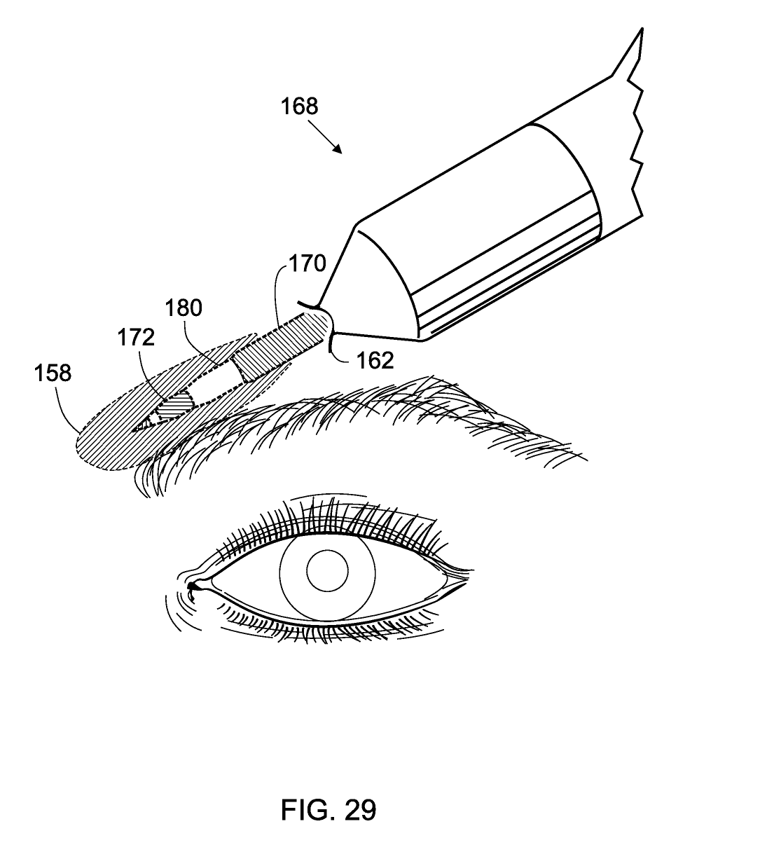

The left side corrugator supercilii muscle 158 (shown in FIG. 29) is selected to illustrate how to use the myoablation system of this embodiment to perform a myoablation procedure. The goal of the following myoablation procedure is to reduce left side corrugator supercilii muscle 158 activity.

The myoablation procedure is accomplished in three steps as follows:

(1) Local anesthesia procedure.

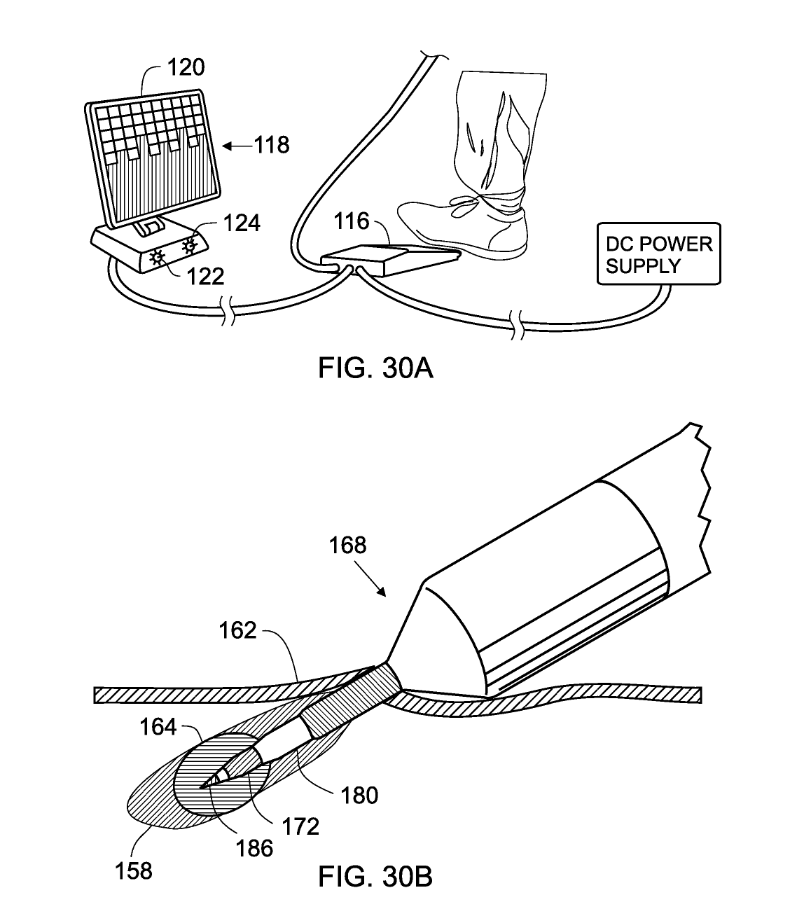

(2) Percutaneous muscle detection (FIGS. 29 and 30A)

(3) Increasing the internal energy in the muscle detected (FIGS. 30A and 30B)

FIG. 28 shows an operational view of the myoablation system shown in FIG. 18. Handle 102 shown in FIG. 28 is hold like a pencil. Handle 102 in connected by mix cable 114 to switch 116 (not shown). Energize indicator 104 green LED lights when radio frequency energy needle electrode tip 168 is being energized. Time indicator 106 yellow LED blinks (at a 2 Hz blinking rate) when radio frequency energy needle electrode tip 168 is being energized.

FIG. 29 shows a schematic view of a myoablation procedure. Local anesthesia procedure is performed as previously described (FIG. 15). Then radio frequency energy needle electrode tip 168 is inserted percutaneously and directed toward left side corrugator supercilii muscle 158 in such a way that accessory conductor 172 is in left side corrugator supercilii muscle 158, and coaxial return electrode 170 is in contact with skin 162. Accessory conductor 172 pickups left side corrugator supercilii muscle 158 electrical signal. Coaxial return electrode 170 provides an electrical return path. Insulator 180 provides insulation.

FIGS. 30A and 30B shows a schematic view of a myoablation procedure. The trace in graphic display 120 of muscle detector 118 indicates that accessory conductor 172 of radio frequency energy needle electrode tip 168 is in left side corrugator supercilii muscle 158. Then switch 116 is pressed and radio frequency energy is delivered through accessory conductor 172 and main conductor 186 in left side corrugator supercilii muscle 158. Radio frequency energy increases the internal energy of muscle tissue resulting in a well-demarcated area of defunctionalized muscle (corrugator supercilii muscle ablated area 164). Next, the patient is asked to frown to determine the degree of reduction of left side corrugator supercilii muscle 158 activity. The procedure is repeated until the desire effect is achieved. In most cases 10 sets of 30 seconds in duration each suffice to achieve the desired degree of reduction of left side corrugator supercilii muscle 158 activity.

Since typical voltage of an intact left side corrugator supercilii muscle 158 is 5.2 millivolts, gain control 122 knob is set at maximum gain (full clockwise turn, gain ratio 1:1011). Sweep time control 124 knob is set at maximum speed (full clockwise turn, sweep frequency of 500 Hz).

Radio frequency energy delivered through accessory conductor 172 and main conductor 186 has a frequency of 2.5 MHz, and a peak-to-peak voltage of 8.4 volts. Radio frequency energy passes through the electrical resistance of the muscle tissue, increasing the internal energy of left side corrugator supercilii muscle 158. This produces a disruption of the muscle contractile proteins actin and myosin, creating an area of defunctionalized muscle (corrugator supercilii muscle ablated area 164). Insulator 180 limits the conduction of radiofrequency energy delivered by accessory conductor 172 and main conductor 186, therefore skin 162 is undamaged.

### Description

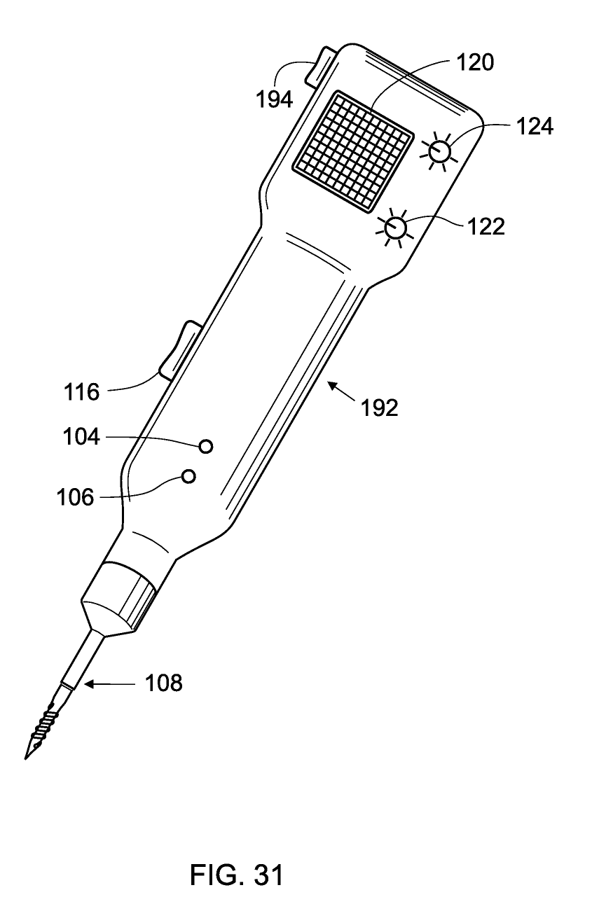

### Third Embodiment—FIGS. 31, 32, 33, 34

FIG. 31 shows a perspective view of the myoablation system of the third embodiment. The myoablation system of FIG. 31 is a wireless version of the myoablation system. The myoablation system of FIG. 31 comprises a hand-held component 192 and detachable thermal needle electrode tip 108. Hand-held component 192 comprises an on-off switch 194, graphic display 120, gain control 122, sweep time control 124, switch 116, energize indicator 104, and time indicator 106.

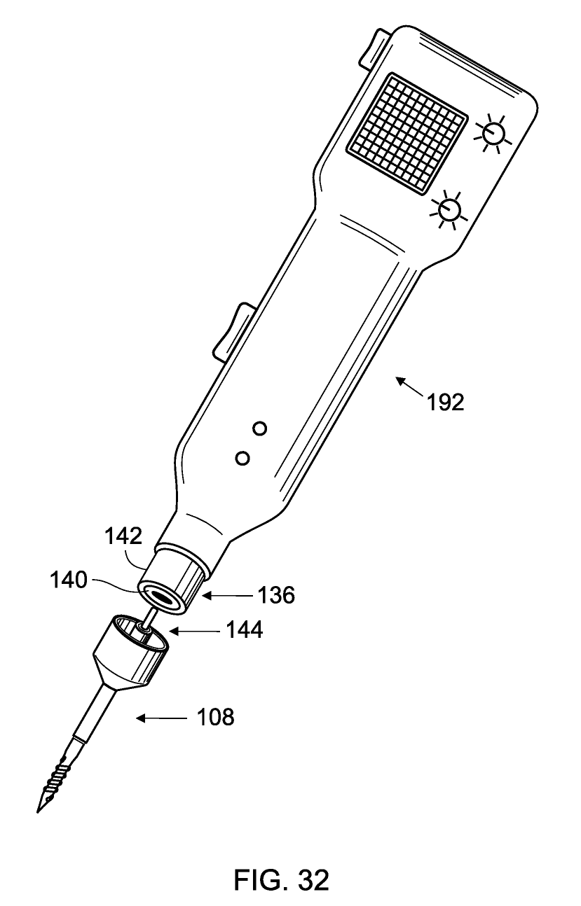

FIG. 32 shows a perspective view of the myoablation system shown in FIG. 31. Thermal needle electrode tip 108 is showing detached from Hand-held component 192. Hand-held component 192 has coaxial jack 136. Coaxial jack 136 has central conductor 138 (not visible), middle conductor 140, and outer conductor 142. Thermal needle electrode tip 108 has coaxial plug 144.

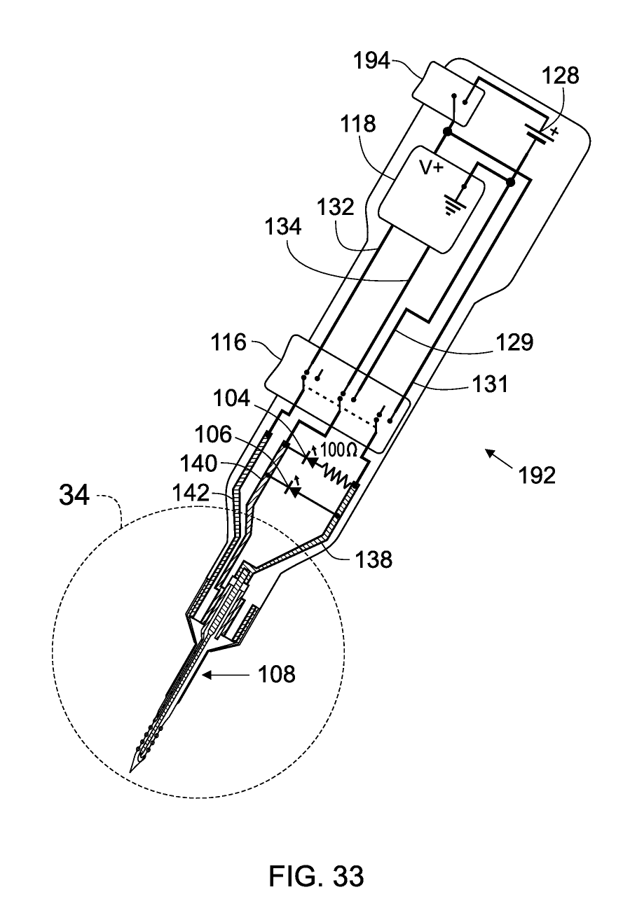

FIG. 33 shows a schematic sectional view of the myoablation system shown in FIG. 31. Hand-held component 192 is attached to thermal needle electrode tip 108.

Muscle detector 118 is connected by sampling ground 132 wire, and by sampling input 134 wire to switch 116 terminals.

Power supply 128 is connected by negative wire 129 to both muscle detector 118 and switch 116. Power supply 128 is connected to on-off switch 194. On-off switch 194 is connected by positive wire 131 to both muscle detector 118 and switch 116.

Switch 116 is connected to central conductor 138. Additionally, switch 116 is connected to middle conductor 140. Similarly, switch 116 is connected to outer conductor 142.

Energize indicator 104 is connected to central conductor 138, and to middle conductor 140. Likewise, time indicator 106 is connected to central conductor 138, and to middle conductor 140.

I contemplate that hand-held component 192 of this embodiment is made of plastic, but others materials are also suitable.

I propose that on-off switch 194 comprises a single pole single throw (push-to-make) on-off switch, but others components are also suitable.

I presently contemplate that switch 116 of this embodiment comprises of a triple pole triple throw momentary (push-to-make) microswitch. However others components for alternating between two or more circuit configurations are also suitable, such as reed switch, tilt switch, transistor, relay, computer-controlled switching mechanism, optoelectronic mechanism, nanotechnology mechanism, molecular mechanism, etc.

I presently propose an electronic version of muscle detector 118 comprising the circuitry shown in FIG. 10. Notwithstanding others components for detecting muscle tissue are also suitable, such as an electromyograph, an oscilloscope, a PC-based oscilloscope, a muscle stimulator, an endoscope, etc.

I currently contemplate that power supply 128 of this embodiment comprises a 9 volt alkaline battery. Nevertheless other power sources are also suitable, such as a rechargeable battery, electrochemical cell, thermoelectric power generator, etc.

When switch 116 is undisturbed, the signal coming from thermal needle electrode tip 108 travels by outer conductor 142, and middle conductor 140 to switch 116; then this signal travels from switch 116 by sampling ground 132 wire, and sampling input 134 wire to muscle detector 118.

When switch 116 is pressed, the electrical energy coming from power supply 128 travels by positive wire 131, and negative wire 129 to switch 116; then this electrical energy travels from switch 116 by central conductor 138, and middle conductor 140 to thermal needle electrode tip 108. At the same time, energize indicator 104 green LED and time indicator 106 yellow LED light. Time indicator 106 yellow LED blinks at a 2 Hz blinking rate, allowing the physician to calculate the time thermal needle electrode tip 108 remains energized.

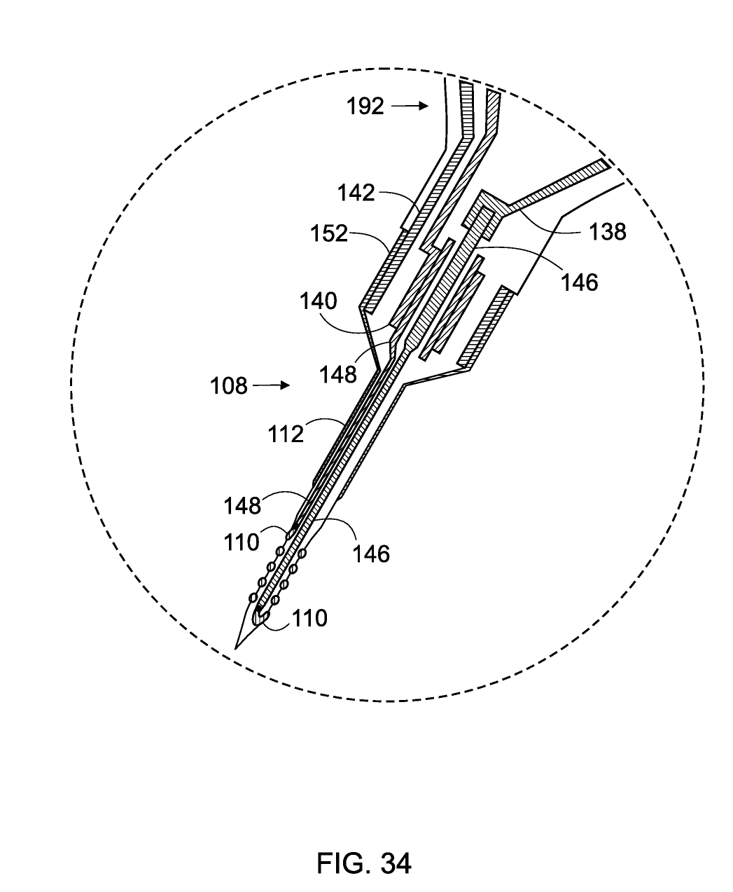

Connection between hand-held component 192 and thermal needle electrode tip 108 is shown enlarged in FIG. 34 for clarity.

FIG. 34 shows an enlarged sectional view of the myoablation system shown in FIG. 33. Hand-held component 192 is attached to thermal needle electrode tip 108. Central conductor 138 is connected by a central rod 146 to resistive heating element 110. Resistive heating element 110 is connected by a middle rod 148 to middle conductor 140. Outer conductor 142 is connected by an outer rod 152 to percutaneous return electrode 112.

### Operation

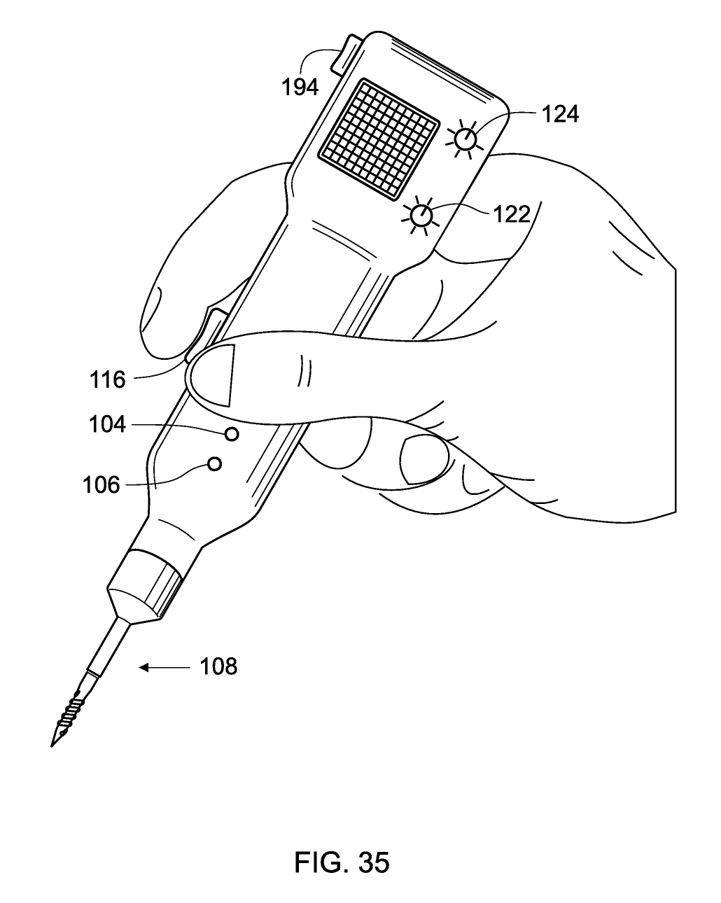

### Third Embodiment—FIGS. 35, 36, 37A, 37B

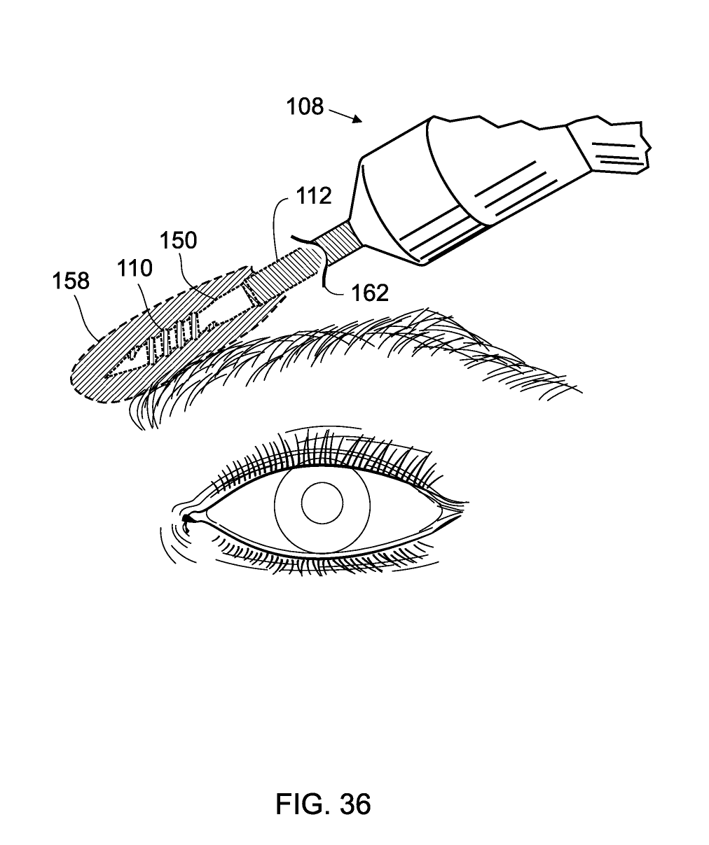

The left side corrugator supercilii muscle 158 (shown in FIG. 36) is selected to illustrate how to use the myoablation system of this embodiment to perform a myoablation procedure. The goal of the following myoablation procedure is to reduce left side corrugator supercilii muscle 158 activity.

The myoablation procedure is accomplished in three steps as follows:

(1) Local anesthesia procedure.

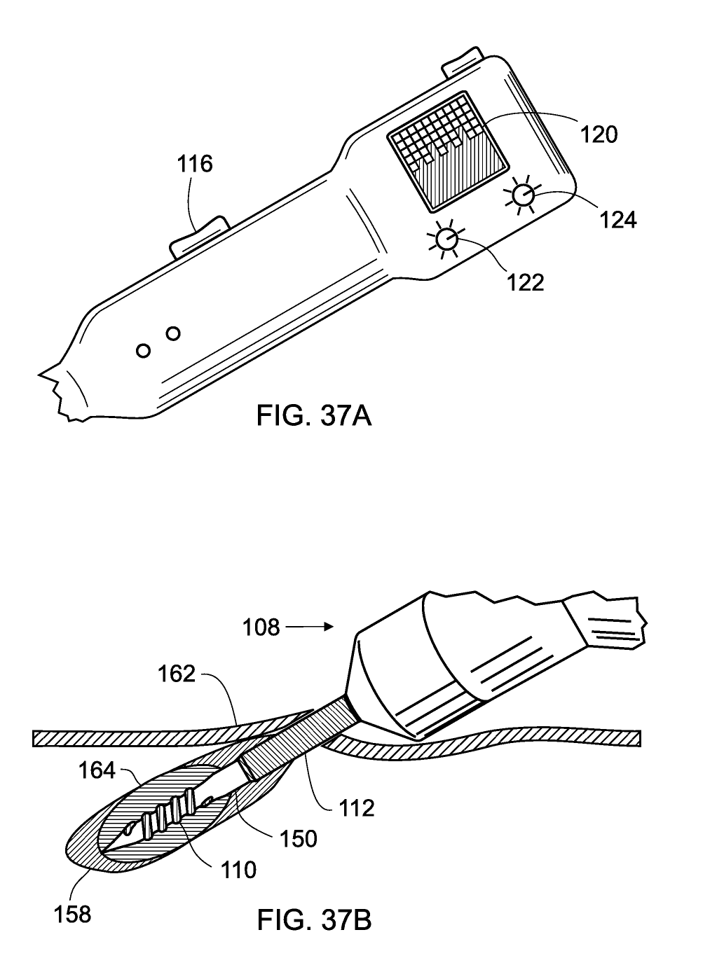

(2) Percutaneous muscle detection (FIGS. 36 and 37A)

(3) Increasing the internal energy in the muscle detected (FIGS. 37A and 37B)

FIG. 35 shows an operational view of the myoablation system shown in FIG. 31. On-off switch 194 is pressed to turn on the system. Myoablation system shown in FIG. 35 is hold like a pencil. When switch 116 is pressed energize indicator 104 and time indicator 106 light. Energize indicator 104 green LED lights when thermal needle electrode tip 108 is being energized. Time indicator 106 yellow LED blinks (at a 2 Hz blinking rate) when thermal needle electrode tip 108 is being energized. Since typical voltage of an intact left side corrugator supercilii muscle 158 is 5.2 millivolts, gain control 122 knob is set at maximum gain (full clockwise turn, gain ratio 1:1011). Sweep time control 124 knob is set at maximum speed (full clockwise turn, sweep frequency of 500 Hz).

FIG. 36 shows a schematic view of a myoablation procedure. Local anesthesia procedure is performed as previously described (FIG. 15). Then thermal needle electrode tip 108 is inserted percutaneously and directed toward left side corrugator supercilii muscle 158 in such a way that resistive heating element 110 is in left side corrugator supercilii muscle 158, and percutaneous return electrode 112 is in contact with skin 162. Resistive heating element 110 pickups left side corrugator supercilii muscle 158 electrical signal. Percutaneous return electrode 112 provides an electrical return path. Electrothermal insulator matrix 150 provides electrical and thermal insulation.

FIGS. 37A and 37B shows a schematic view of a myoablation procedure. The trace in graphic display 120 indicates that resistive heating element 110 of thermal needle electrode tip 108 is in left side corrugator supercilii muscle 158. Then switch 116 is pressed and heat is delivered through resistive heating element 110 in left side corrugator supercilii muscle 158. Heat increases the internal energy of muscle tissue resulting in a well-demarcated area of defunctionalized muscle (corrugator supercilii muscle ablated area 164). Next, the patient is asked to frown to determine the degree of reduction of left side corrugator supercilii muscle 158 activity. The procedure is repeated until the desire effect is achieved.

Since typical voltage of an intact left side corrugator supercilii muscle 158 is 5.2 millivolts, gain control 122 knob is set at maximum gain (full clockwise turn, gain ratio 1:1011). Sweep time control 124 knob is set at maximum speed (full clockwise turn, sweep frequency of 500 Hz).

Electrothermal insulator matrix 150 limits the conduction of heat generated by resistive heating element 110. The temperature of percutaneous return electrode 112 in contact with skin 162 is 37.5° C., therefore skin 162 is undamaged.

### Description

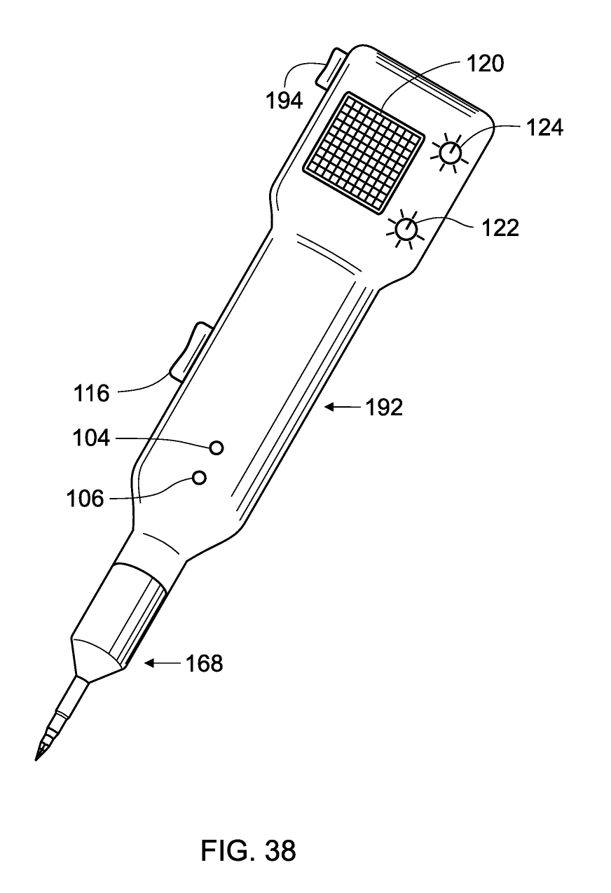

### Fourth Embodiment—FIGS. 38, 39, 40, 41

FIG. 38 shows a perspective view of the myoablation system of the fourth embodiment. The myoablation system of FIG. 38 is another wireless version of the myoablation system. The myoablation system of FIG. 38 comprises hand-held component 192 and detachable radio frequency energy needle electrode tip 168. Hand-held component 192 comprises on-off switch 194, graphic display 120, gain control 122, sweep time control 124, switch 116, energize indicator 104, and time indicator 106.

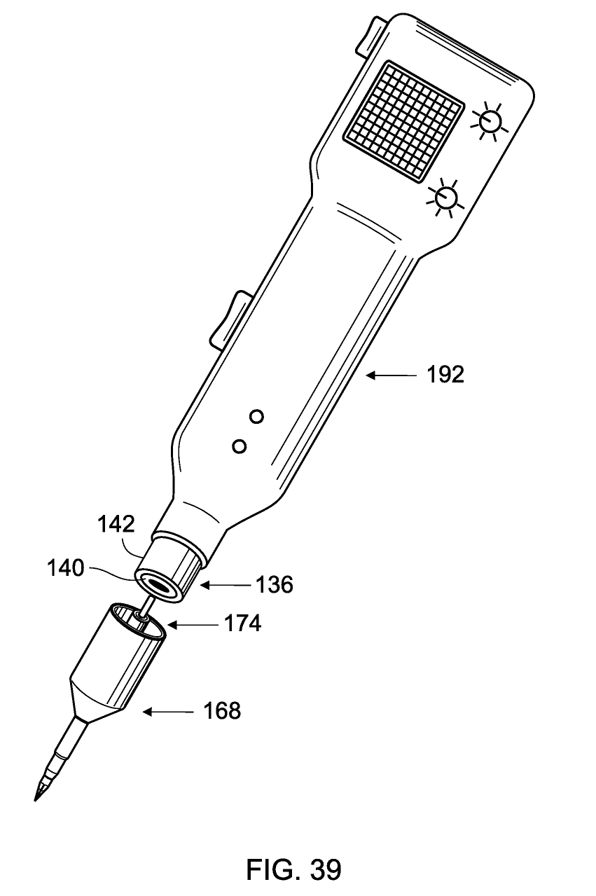

FIG. 39 shows a perspective view of the myoablation system shown in FIG. 38. Radio frequency energy needle electrode tip 168 is showing detached from Hand-held component 192. Hand-held component 192 has coaxial jack 136. Coaxial jack 136 has central conductor 138 (not visible), middle conductor 140, and outer conductor 142. Radio frequency energy needle electrode tip 168 has coaxial connector 174.

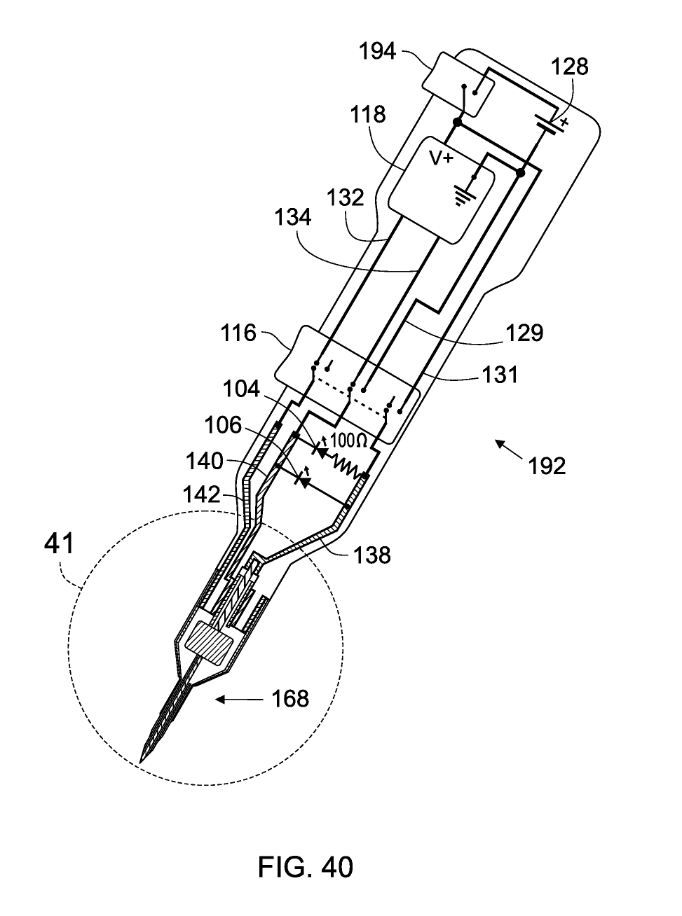

FIG. 40 shows a schematic sectional view of the myoablation system shown in FIG. 38. Hand-held component 192 is attached to radio frequency energy needle electrode tip 168.

Muscle detector 118 is connected by sampling ground 132 wire, and by sampling input 134 wire to switch 116 terminals.

Power supply 128 is connected by negative wire 129 to both muscle detector 118 and switch 116. Power supply 128 is connected to on-off switch 194. On-off switch 194 is connected by positive wire 131 to both muscle detector 118 and switch 116.

Switch 116 is connected to central conductor 138. Additionally, switch 116 is connected to middle conductor 140. Similarly, switch 116 is connected to outer conductor 142.

Energize indicator 104 is connected to central conductor 138, and to middle conductor 140. Likewise, time indicator 106 is connected to central conductor 138, and to middle conductor 140.

When switch 116 is undisturbed, the signal coming from radio frequency energy needle electrode tip 168 travels by outer conductor 142, and middle conductor 140 to switch 116; then this signal travels from switch 116 by sampling ground 132 wire, and sampling input 134 wire to muscle detector 118.

When switch 116 is pressed, the electrical energy coming from power supply 128 travels by positive wire 131, and negative wire 129 to switch 116; then this electrical energy travels from switch 116 by central conductor 138, and middle conductor 140 to radio frequency energy needle electrode tip 168. At the same time, energize indicator 104 green LED and time indicator 106 yellow LED light. Time indicator 106 yellow LED blinks at a 2 Hz blinking rate, allowing the physician to calculate the time radio frequency energy needle electrode tip 168 remains energized.

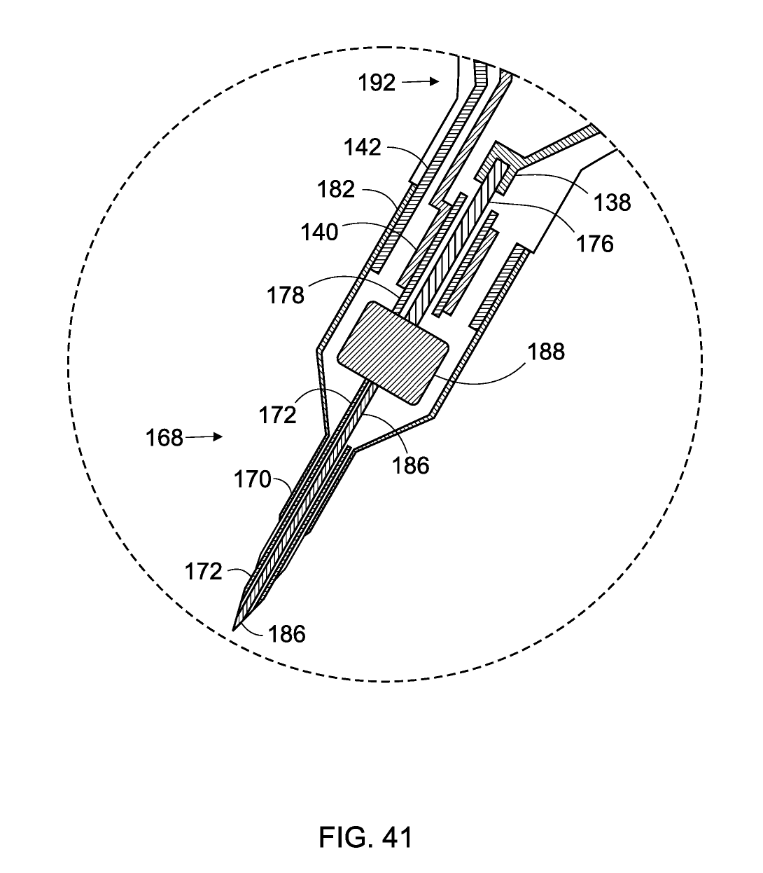

Connection between hand-held component 192 and radio frequency energy needle electrode tip 168 is shown enlarged in FIG. 41 for clarity.

FIG. 41 shows an enlarged partial view of the myoablation system shown in FIG. 40. Hand-held component 192 is attached to radio frequency energy needle electrode tip 168. Central conductor 138 is connected by central coaxial conductor 176 to radio frequency energy generator 188. Radio frequency energy generator 188 is connected by middle coaxial conductor 178 to middle conductor 140. Outer conductor 142 is connected by outer coaxial conductor 182 to coaxial return electrode 170.

Radio frequency energy generator 188 is connected to accessory conductor 172. In addition, radio frequency energy generator 188 is connected to main conductor 186.

### Operation

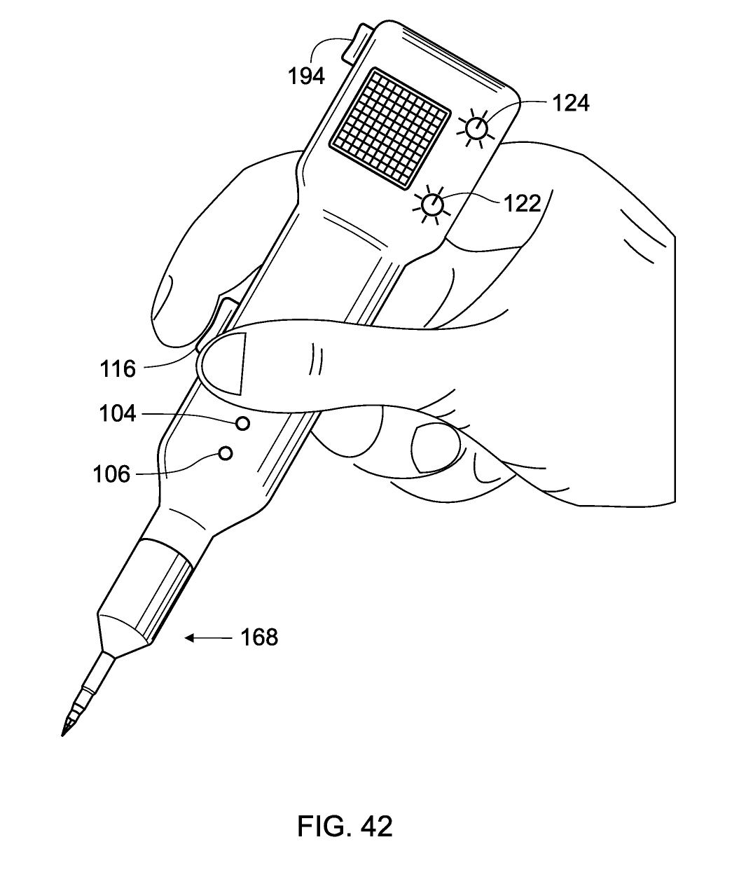

### Fourth Embodiment—FIGS. 42, 43, 44A, 44B

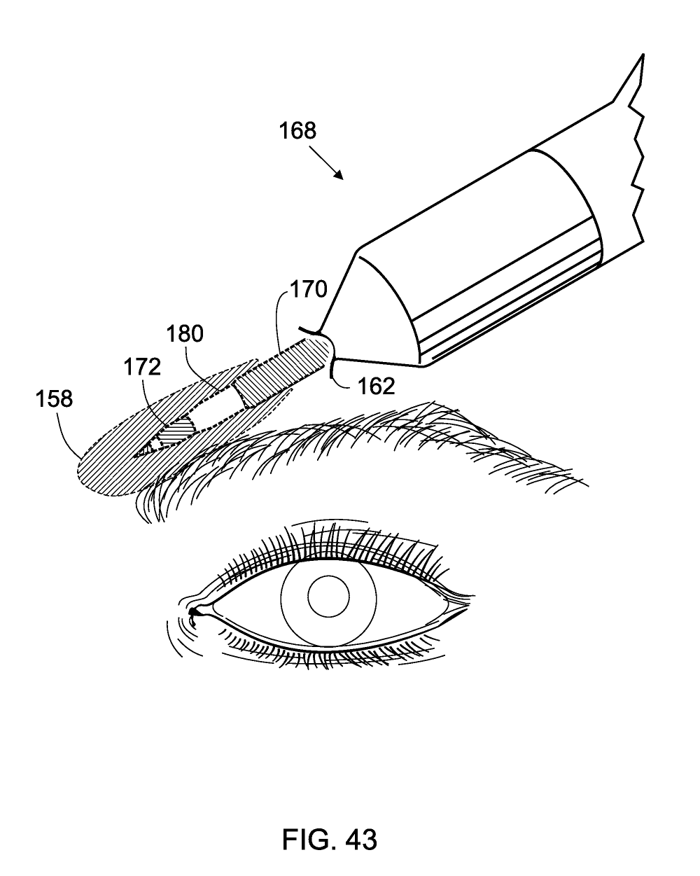

The left side corrugator supercilii muscle 158 (shown in FIG. 43) is selected to illustrate how to use the myoablation system of this embodiment to perform a myoablation procedure. The goal of the following myoablation procedure is to reduce left side corrugator supercilii muscle 158 activity.

The myoablation procedure is accomplished in three steps as follows:

(1) Local anesthesia procedure.

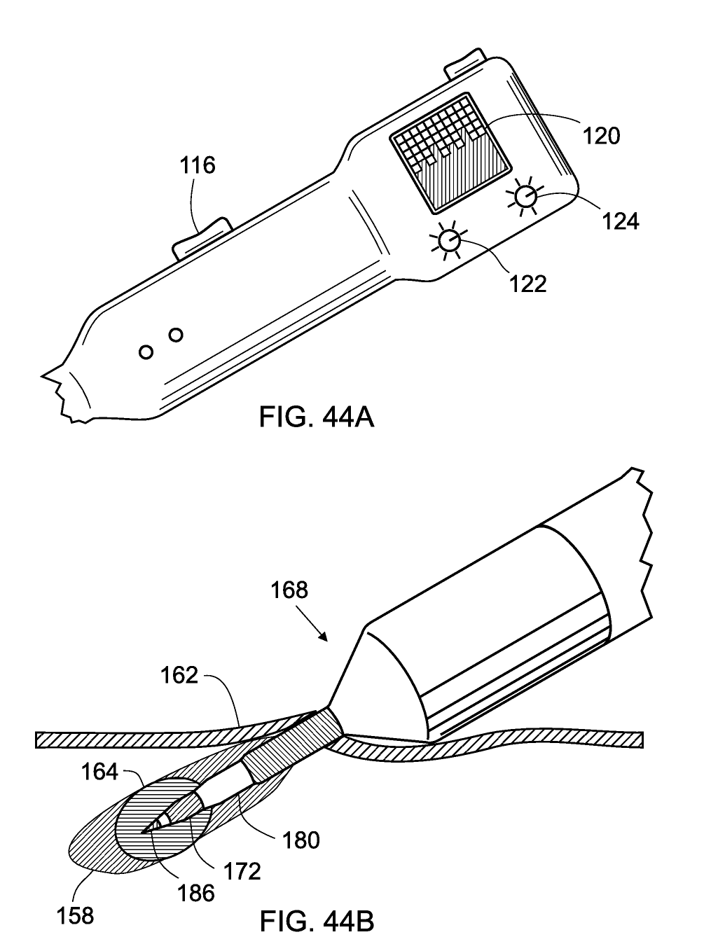

(2) Percutaneous muscle detection (FIGS. 43 and 44A)

(3) Increasing the internal energy in the muscle detected (FIGS. 44A and 44B)

FIG. 42 shows an operational view of the myoablation system shown in FIG. 38. On-off switch 194 is pressed to turn on the system. Myoablation system shown in FIG. 42 is hold like a pencil. When switch 116 is pressed energize indicator 104 and time indicator 106 light. Energize indicator 104 green LED lights when radio frequency energy needle electrode tip 168 is being energized. Time indicator 106 yellow LED blinks (at a 2 Hz blinking rate) when radio frequency energy needle electrode tip 168 is being energized. Since typical voltage of an intact left side corrugator supercilii muscle 158 is 5.2 millivolts, gain control 122 knob is set at maximum gain (full clockwise turn, gain ratio 1:1011). Sweep time control 124 knob is set at maximum speed (full clockwise turn, sweep frequency of 500 Hz).

FIG. 43 shows a schematic view of the myoablation procedure. Local anesthesia procedure is performed as previously described (FIG. 15). Then radio frequency energy needle electrode tip 168 is inserted percutaneously and directed toward left side corrugator supercilii muscle 158 in such a way that accessory conductor 172 is in left side corrugator supercilii muscle 158, and coaxial return electrode 170 is in contact with skin 162. Accessory conductor 172 pickups left side corrugator supercilii muscle 158 electrical signal. Coaxial return electrode 170 provides an electrical return path. Insulator 180 provides insulation.

FIGS. 44A and 44B shows a schematic view of a myoablation procedure. The trace in graphic display 120 indicates that accessory conductor 172 of radio frequency energy needle electrode tip 168 is in left side corrugator supercilii muscle 158. Then switch 116 is pressed and radio frequency energy is delivered through accessory conductor 172 and main conductor 186 in left side corrugator supercilii muscle 158. Radio frequency energy increases the internal energy of muscle tissue resulting in a well-demarcated area of defunctionalized muscle (corrugator supercilii muscle ablated area 164). Next, the patient is asked to frown to determine the degree of reduction of left side corrugator supercilii muscle 158 activity. The procedure is repeated until the desire effect is achieved.

Since typical voltage of an intact left side corrugator supercilii muscle 158 is 5.2 millivolts, gain control 122 knob is set at maximum gain (full clockwise turn, gain ratio 1:1011). Sweep time control 124 knob is set at maximum speed (full clockwise turn, sweep frequency of 500 Hz).

Radio frequency energy delivered through accessory conductor 172 and main conductor 186 has a frequency of 2.5 MHz, and a peak-to-peak voltage of 8.4 volts. Radio frequency energy passes through the electrical resistance of the muscle tissue, increasing the internal energy of left side corrugator supercilii muscle 158. This produces a disruption of the muscle contractile proteins actin and myosin, creating an area of defunctionalized muscle (corrugator supercilii muscle ablated area 164). Insulator 180 limits the conduction of radiofrequency energy delivered by accessory conductor 172 and main conductor 186, therefore skin 162 is undamaged.

### Description

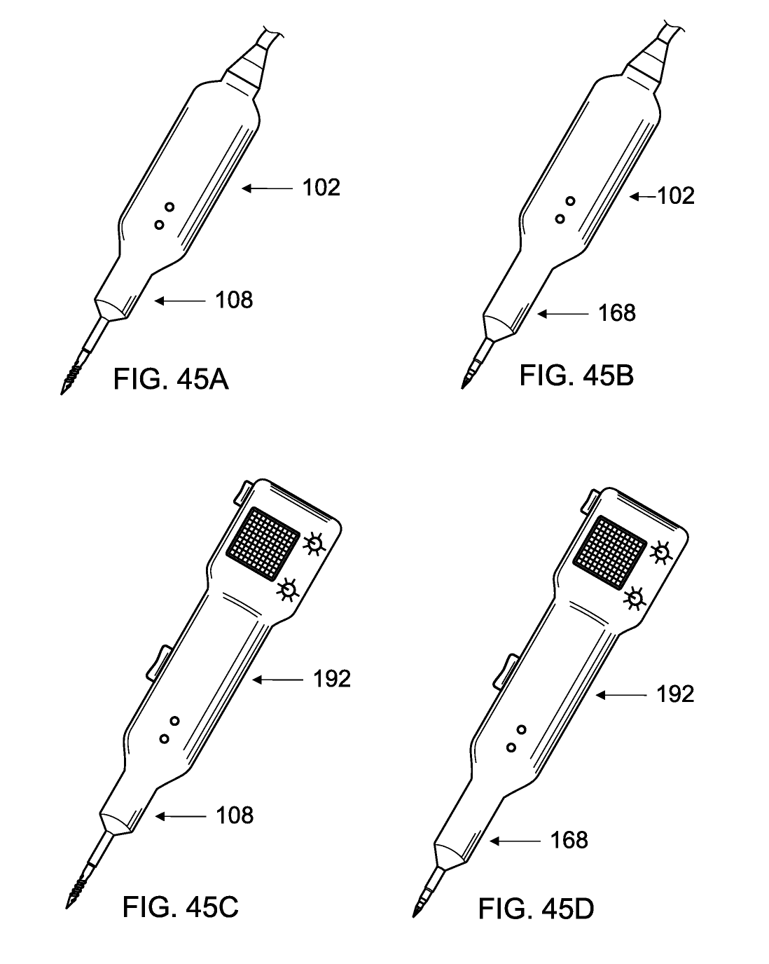

### Fifth Embodiment—FIG. 45A

FIG. 45A shows a partial view of the myoablation system of the fifth embodiment. This embodiment is similar to the myoablation system of FIG. 1. However myoablation system of FIG. 45A comprises a handle 102 with a non-detachable thermal needle electrode tip 108.

### Operation

### Fifth Embodiment

The operation of the myoablation system of FIG. 45A is similar to the operation of the myoablation system of FIG. 1.

### Description

### Sixth Embodiment—FIG. 45B

FIG. 45B shows a partial view of the myoablation system of the sixth embodiment. This embodiment is similar to the myoablation system of FIG. 18. However myoablation system of FIG. 45B comprises a handle 102 with a non-detachable radio frequency energy needle electrode tip 168.

### Operation

### Sixth Embodiment

The operation of the myoablation system of FIG. 45B is similar to the operation of the myoablation system of FIG. 18.

### Description

### Seventh Embodiment—FIG. 45C

FIG. 45C shows a perspective view of the myoablation system of the seventh embodiment. The myoablation system of FIG. 45C is another wireless version of the myoablation system. This embodiment is similar to the myoablation system of FIG. 31. However myoablation system of FIG. 45C comprises a hand-held component 192 with a non-detachable thermal needle electrode tip 108.

### Operation

### Seventh Embodiment

The operation of the myoablation system of FIG. 45C is similar to the operation of the myoablation system of FIG. 31.

### Description

### Eighth Embodiment—FIG. 45D

FIG. 45D shows a perspective view of the myoablation system of the eight embodiment. The myoablation system of FIG. 45D is another wireless version of the myoablation system. This embodiment is similar to the myoablation system of FIG. 38. However myoablation system of FIG. 45D comprises a hand-held component 192 with a non-detachable radio frequency energy needle electrode tip 168.

### Operation

### Eighth Embodiment

The operation of the myoablation system of FIG. 45D is similar to the operation of the myoablation system of FIG. 38.

### ADVANTAGES

From the description above, a number of advantages of some embodiments of the myoablation system become evident:

(a) Safety. The myoablation system devitalizes a facial muscle by increasing its internal energy, thus avoiding the need to charring tissues to produce the desired effect. No high-energy current pass through the patient's body, thus minimizing the risk of liability issues. The whole procedure is performed under local anesthesia. Mayor surgical and anesthetic risks are avoided. Disposable, sterilized tips can be manufactured at a very low cost. Neither neurotoxins nor neurotoxin implants enter the patient's body.

(b) Extensive clinical applications. The myoablation system can be employed for the treatment of migraine, facial paralysis, spasmodic torticollis, blepharospasm, aesthetic enhancement, etc. The myoablation system can be used for percutaneous ablation of skeletal muscle tissue, smooth muscle tissue, peripheral nerve tissue, etc

(c) Ease of use. The myoablation system is focused in the facial muscles rather than in the facial nerves. The myoablation system simple LED visual indicators are easy to read.

(d) Easy of production. The myoablation system requires minimal engineering for manufacturing. The myoablation system components are inexpensive and ready available. The myoablation system does not require sophisticated production facilities.

### CONCLUSION, RAMIFICATIONS, AND SCOPE

While the above description contains many specificities, these should not be constructed as limitations on the scope of any embodiment, but as exemplifications of the presently embodiments thereof. Many other ramifications and variations are possible within the reachings of the various embodiments.

For example, muscle detector 118 can be eliminated. A highly skilled physician could perform a myoablation procedure using a myoablation system without muscle detector 118. Muscle detector 118 can be replaced by a combination of a computer-based oscilloscope and a muscle stimulator. Muscle detector 118 can be replaced by a computer with appropriate software. Muscle detector 118 can be replaced by an oscilloscope. Muscle detector 118 can be replaced by a computer-based oscilloscope. Muscle detector 118 and power supply 128 can be assembled in a single box.

Switch 116 can be incorporated in handle 102. Mix cable 114, power cable 130, and input cable 126 can be replaced by detachable cables. Thermal needle electrode tip 108 can be modified to deliver other type of energy (i.e. ultrasound, mechanical, shock waves, micro shock waves, microwaves, laser, light, plasma, ionization, radiation, gamma rays, etc). Percutaneous return electrode 112 can be replaced by a return electrode plate. Coaxial plug 144 can be replaced by a multi-axial connector. Coaxial jack 136 can be replaced by a multi-axial connector.

Radio frequency energy needle electrode tip 168 can be modified to deliver other type of energy (i.e. ultrasound, mechanical, shock waves, micro shock waves, microwaves, laser, light, plasma, ionization, radiation, gamma rays, etc). Coaxial connector 174 can be replaced by a multi-axial connector. Coaxial connector 174 can be replaced by a multi-axial connector. Radio frequency energy generator 188 can be modified to generate other type of energy (i.e. ultrasound, mechanical, shock waves, micro shock waves, microwaves, laser, light, plasma, ionization, radiation, gamma rays, etc).

Radio frequency energy generator 188 can be replaced by a micromotor connected with a thin drill bit. This drill bit can be inserted percutaneously to perform a mechanical ablation of muscle tissue.

The myoablation system can be used for percutaneous ablation of skeletal muscle tissue, smooth muscle tissue, peripheral nerve tissue, etc.

Thus the scope of the embodiments should be determined by the appended claims and their legal equivalents, and not by the examples given.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A myoablation system for modifying the facial dynamics, comprising:
means for increasing the internal energy in a part of a facial muscle via the percutaneous route;
means for alternating between two or more circuit configurations, operably connected to said means for increasing the internal energy in a part of a facial muscle via the percutaneous route; and
means for providing power, operably connected to said means for alternating between two or more circuit configurations.

2. The myoablation system in accordance with claim 1, wherein said means for increasing the internal energy in a part of a facial muscle via the percutaneous route comprises a hand piece, having a tip.

3. The myoablation system in accordance with claim 1, wherein said means for alternating between two or more circuit configurations comprises a switch.

4. The myoablation system in accordance with claim 1, wherein said means for providing power comprises a power supply.

5. A myoablation system for modifying the facial dynamics, comprising:
a hand piece, having a tip, for increasing the internal energy in a part of a facial muscle via the percutaneous route;
a switch, for alternating between two or more circuit configurations, operably connected to said hand piece; and
a power supply, for providing power, operably connected to said switch.

6. The myoablation system as recited in claim 5, further comprising:
a muscle detector, for detecting muscle tissue, operably connected to said switch.

7. A myoablation system for modifying the facial dynamics, comprising:
a hand piece, having a tip, for increasing the internal energy in a part of a facial muscle via the percutaneous route;
a switch, for alternating between two or more circuit configurations, operably connected to said hand piece;
a power supply, for providing power, operably connected to said switch; and
a muscle detector, for detecting muscle tissue, operably connected to said switch.
